(19) United States
(12) Patent Application Publication
(10) Pub. No.: US 2026/0009043 A1
(43) Pub. Date: Jan. 8, 2026

(54) METHOD FOR PRODUCING SPERMIDINE

(71) Applicant: Zhuo Hong Chaoyuan Biotechnology (Wuxi) Co., Ltd, Wuxi (CN)

(72) Inventors: Yujie CAI, Wuxi (CN); Linbo GOU, Wuxi (CN); Di LIU, Wuxi (CN); Yi YAN, Wuxi (CN); Jie WANG, Wuxi (CN); Xiaoyuan ZHANG, Wuxi (CN); Yanrui DING, Wuxi (CN)

(21) Appl. No.: 19/330,932

(22) Filed: Sep. 17, 2025

Related U.S. Application Data
(63) Continuation of application No. PCT/CN2024/119608, filed on Sep. 19, 2024.

(30) Foreign Application Priority Data
Sep. 20, 2023 (CN) 202311230139.0

(51) Int. Cl. C12N 15/70; C12N 9/06; C12N 9/78; C12N 9/88; C12N 15/74

(52) U.S. Cl. CPC C12N 15/70; C12N 9/0028; C12N 9/78; C12N 9/88; C12N 15/74

(57) ABSTRACT

The present disclosure belongs to the technical field of synthetic biology, and relates to a method for producing spermidine. The present disclosure adopts a metabolic engineering method by introducing a novel exogenous pathway, eliminates the inhibition of various products in the spermidine synthesis pathway, changes the spermidine transport system, and enhance the enzymatic activities of key pathways. As a result, genetically engineered strains of Serratia marcescens and Escherichia coli with high yield of spermidine are obtained. The spermidine concentration in the shake flask supernatant reaches 26.8 g/L for genetically engineered strain of Serratia marcescens and 24.7 g/L for the genetically engineered strain of Escherichia coli, respectively, both demonstrating good potential for industrial application.

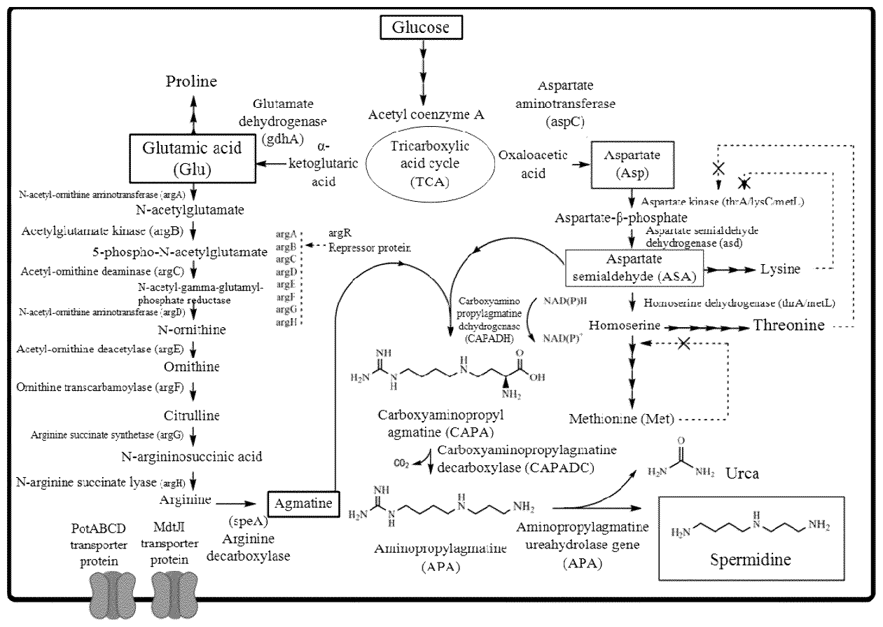

### REFERENCE TO SEQUENCE LISTING

The instant application contains a Sequence Listing in XML format as a file named “PC240006A.xml”, created on 2025 Sep. 11, of 325,614 byte in size, and which is hereby incorporated by reference in its entirety.

### TECHNICAL FIELD

The present disclosure belongs to the technical field of synthetic biology, and relates to a method for producing spermidine.

### BACKGROUND

Spermidine, with a molecular formula of NH₂(CH₂)₃NH(CH₂)₄NH₂, is an important biologically active substance that is widely present in microorganisms, plants, and animals. Spermidine is a multifunctional polyamine that plays a crucial role in a variety of biological processes, including cell proliferation, differentiation, DNA stability, and cellular stress responses. In industrial applications, spermidine is often used as a catalyst in the production of some plastics and polymers. In the agricultural field, it serves as a plant growth regulator.

Despite the wide applications of spermidine in industry and agriculture, traditional chemical synthesis methods for spermidine have numerous defects. The traditional methods usually require the use of highly toxic chemical reagents, posing safety risks to operators and resulting in environmental pollution and waste disposal problems. In addition, chemical synthesis usually involves multiple reaction steps, which are complicated and inefficient, resulting in relatively low yield.

Compared with chemical synthesis, microbial synthesis of spermidine has some noticeable advantages. It is generally environmentally friendly and does not require the use of highly toxic reagents, thereby reducing a negative impact on environment. Moreover, microbial synthesis may be carried out under mild conditions, reducing energy consumption and production costs. Most importantly, microorganisms have diverse metabolic pathways that enable the synthesis of spermidine from naturally occurring metabolites. However, existing microbial production methods also face limitations. The stability of production strains is a critical issue, especially during prolonged fermentation processes, microorganisms may lose their ability to synthesize spermidine. In addition, the spermidine yield is often inadequate, requiring improvement through metabolic engineering approaches. Furthermore, the fermentation process may be complex and require careful design and optimization.

Therefore, improving spermidine yield in microbial fermentation is one of the solutions, involving genetically engineered microorganisms to enhance their ability to increase the yield of spermidine.

### SUMMARY

The present disclosure reveals that Serratia marcescens exhibits excellent growth rate and metabolic adaptability, making it able to grow under various nutrient sources and environmental conditions. Moreover, a metabolic network of Serratia marcescens has been naturally adapted for polyamine synthesis, providing it with an inherent advantage for production. Compared with traditional Escherichia coli (1.5 g/L), Serratia marcescens demonstrates significantly higher tolerance to spermidine (up to 7 g/L), which is conducive to high-concentration spermidine production. Therefore, the present disclosure provides a genetically engineered strain of Serratia marcescens and a genetically engineered strain of Escherichia coli (E. coli), each of which introduces a novel exogenous synthetic pathway for spermidine synthesis, allowing for high yield of spermidine using Serratia marcescens or E. coli as hosts.

A first objective of the present disclosure is to provide a genetically engineered strain, which introduces an exogenous spermidine synthetic pathway, and contains genes encoding carboxyaminopropylagmatine dehydrogenase (CAPADH), carboxyaminopropylagmatine decarboxylase (CAPADC), and aminopropylagmatine ureahydrolase (APAUH).

In one embodiment, the genetically engineered strain is a genetically engineered strain of Serratia marcescens or a genetically engineered strain of E. coli.

Optionally, the genetically engineered strain of E. coli is constructed using E. coli BL21(DE3), E. coli JM109, E. coli DH5α, E. coli Top10, or E. coli MG1655 as hosts.

Optionally, the genetically engineered strain of Serratia marcescens is constructed using Serratia marcescens HBQA7 as a host.

In one embodiment, the CAPADH may be from any suitable source; optionally, it may be carboxyaminopropylagmatine dehydrogenase derived from Spirochaeta thermophila, Denitrovibrio acetiphilus, Geobacter sulfurreducens, Ilumatobacter coccineus and the like. More optionally, it may be derived from Spirochaeta thermophila DSM 6578 or Denitrovibrio acetiphilus DSM 12809 or Geobacter sulfurreducens KN400 or Ilumatobacter coccineus YM16-304.

In one embodiment, the CAPADC may be from any suitable source; optionally, it may be carboxyaminopropylagmatine decarboxylase derived from Spirochaeta thermophila, Denitrovibrio acetiphilus, Geobacter sulfurreducens, Ilumatobacter coccineus and the like. More optionally, it may be derived from Spirochaeta thermophila DSM 6578 or Denitrovibrio acetiphilus DSM 12809 or Geobacter sulfurreducens KN400 or Ilumatobacter coccineus YM16-304.

In one embodiment, the APAUH may be from any suitable source; optionally, it may be aminopropylagmatine ureahydrolase derived from Spirochaeta thermophila, Denitrovibrio acetiphilus, Geobacter sulfurreducens, Ilumatobacter coccineus and the like. More optionally, it may be derived from Spirochaeta thermophila DSM 6578 or Denitrovibrio acetiphilus DSM 12809 or Geobacter sulfurreducens KN400 or Ilumatobacter coccineus YM16-304.

In one embodiment, an argR gene encoding a repressor protein is knocked out from the genetically engineered strain, and an argA negative feedback mutant is constructed.

In one embodiment, the argA negative feedback mutant is a mutant in which C at a position 40 of the argA gene is mutated to T, converting the encoded histidine to tyrosine to significantly increase the yield of arginine.

In one embodiment, the engineered strain further utilizes a strong promoter to replace native promoters of a lysC gene encoding aspartate kinase, an asd gene encoding aspartate semialdehyde dehydrogenase, an argA gene encoding N-acetylglutamate synthase, an argH gene of arginine succinate lyase, and an speA gene encoding arginine decarboxylase. For example, a strong promoter trc is used to replace the native promoters of lysC, asd, argA, argH, and speA to enhance the expression of aspartate kinase, aspartate semialdehyde dehydrogenase, N-acetylglutamate synthase, arginine succinate lyase, and arginine decarboxylase. Optionally, a sequence of the strong promoter is as shown in SEQ ID NO: 187.

In one embodiment, the strong promoter is a highly efficient binding site for RNA polymerase on the template, enabling efficient transcription initiation and thereby enhancing gene transcription efficiency.

In one embodiment, genes encoding the PotABCD transport proteins are knocked out from the genetically engineered strain, thereby reducing the uptake of spermidine into cells; optionally, the expression of mdtJI genes encoding transport proteins responsible for exporting spermidine is further enhanced, for example, by replacing the native promoter of the mdtJI genes with a strong promoter.

In one embodiment, thrA and metL genes encoding homoserine synthase are knocked out from the genetically engineered strain; alternatively, a dapA gene encoding dihydrodipicolinate synthase is further knocked out from the genetically engineered strain on the basis that the thrA and metL genes are knocked out; alternatively, a proC gene encoding pyrroline-5-carboxylate reductase is further knocked out from the genetically engineered strain on the basis that the thrA, metL, and dapA genes are knocked out.

In one embodiment, an astA gene encoding arginine N-succinyltransferase is knocked out from the genetically engineered strain.

In one embodiment, the strong promoter is a trc promoter.

In one embodiment, the genetically engineered strains are integrated and expressed using the principle of homologous recombination.

In one embodiment, gene knockouts are performed using homologous recombination technology.

In one embodiment, gene knockouts are performed using Crispr/Cas9 gene-editing technology.

A second objective of the present disclosure is to provide application of the genetically engineered strain of Serratia marcescens the production of spermidine.

The present disclosure further provides application of the genetically engineered strain of E. coli the production of spermidine, where any of the above-mentioned genetically engineered strains is placed in a glucose-containing culture medium and subjected to fermentation to prepare spermidine.

In one embodiment, the fermentation is carried out at pH 7, 37° C., with a shaking speed of 200 rpm in a shake flask for 24 hours.

In one embodiment, the genetically engineered strain may be used for high-density cell culture to produce spermidine.

The present disclosure further provides a method for increasing spermidine yield, where any of the above-mentioned genetically engineered strains is placed in a glucose-containing culture medium and subjected to fermentation to prepare spermidine.

In one embodiment, the fermentation is carried out at pH 7, 37° C., with a shaking speed of 200 rpm in a shake flask for 24 hours.

In one embodiment, the genetically engineered strain may be used for high-density cell culture to produce spermidine.

The present disclosure further provides a method for modifying a genetically engineered strain. Common primers are used to amplify gene fragments of carboxyaminopropylagmatine dehydrogenase (CAPADH) and carboxyaminopropylagmatine decarboxylase (CAPADC), and a first multiple cloning site of the pBBR1MCS-2 plasmid is digested with HindIII and EcoRV. After purification of the digestion product, the digested plasmid is ligated with the gene fragments of CAPADH and CAPADC to construct a plasmid carrying the genes of CAPADH and CAPADC;
a gene primer of aminopropylagmatine ureahydrolase (APAUH) is used to amplify a gene fragment of APAUH, and a second multiple cloning site of plasmid carrying the CAPADH and CAPADC genes is digested with EcoRI and SpeI. After purification of the digestion product, the digested plasmid is ligated with the gene fragments of APAUH to construct a plasmid carrying genes of APADH, CAPADC, and APAUH.
the plasmid carrying an APADH gene, an CAPADC gene, and an APAUH gene is transformed into JM109 competent cells, positive clones are selected on kanamycin-resistant plates and further verified by colony PCR; and a plasmid is extracted from the JM109 strain and transformed into the competent engineered strain to construct a spermidine-producing strain;
optionally, the competent engineered strain includes E. coli BL21 (DE3), E. coli JM109, E. coli DH5α, E. coli Top10, E. coli MG1655 or Serratia marcescens HBQA7;
optionally, the APADH gene, the CAPADC gene, and the APAUH gene may be cloned from genomes of Spirochaeta thermophila DSM 6578, Denitrovibrio acetiphilus DSM 12809, Geobacter sulfurreducens KN400, and Ilumatobacter coccineus YM16-304; and
optionally, nucleotide sequences of the common primers are as shown in SEQ ID NO: 1-2; and nucleotide sequences of the primers for the APAUH gene are as shown in SEQ ID NO: 3-4.

### Beneficial Effects

The present disclosure constructs a genetically engineered strain of Serratia marcescens and a genetically engineered strain of E. coli capable of producing high spermidine from glucose using a synthetic biology method. The production process is simple, the raw materials are readily available and low-cost. Therefore, the present disclosure has good prospects for industrial application. Specific beneficial effects are as follows:
(1) The present disclosure introduces an exogenous pathway, and realizes a novel synthetic pathway for the synthesis of spermidine in Serratia marcescens by incorporating exogenous APADH gene, exogenous CAPADC gene, and exogenous APAUH gene. Compared with the traditional production pathways, the genetically engineered strain of Serratia marcescens of the present disclosure can significantly improve the production of spermidine. When only the exogenous pathway is introduced, a concentration of spermidine reaches 5.9 g/L.
(2) The present disclosure eliminates the feedback inhibition of arginine and the repression in the genetically engineered strain of Serratia marcescens, and removes the repression of the pathway by ArgR. After optimization, the spermidine yield reaches 8.6 g/L.
(3) The present disclosure enhances the expression of aspartate kinase encoded by the lysC gene and aspartate semialdehyde dehydrogenase encoded by the asd gene in the genetically engineered strain of Serratia marcescens. LysC and asd are key enzymes that catalyze the synthesis of aspartate semialdehyde from aspartate. N-acetylglutamate synthase, and arginine succinate lyase encoded by argA and argH genes are the first and last enzymes in the arginine synthesis pathway, respectively. The arginine decarboxylase encoded by the speA gene is a key enzyme that catalyzes further synthesis of agmatine from arginine. The expression of the five enzymes can further increase the yield of spermidine to 16.3 g/L.
(4) The present disclosure improves the transport pathway of spermidine in the genetically engineered strain of Serratia marcescens. PotABCD and MdtJI proteins are responsible for transporting spermidine into and out of cells, respectively. By knocking out potA and enhancing the expression of mdtJI, the spermidine yield is increased to 21.4 g/L.
(5) After knocking out the thrA, metL, dapA, and proC genes in the genetically engineered strain of Serratia marcescens, the present disclosure can achieve the yield of spermidine up to 23.3 g/L.
(6) After further knocking out the ast series genes in the genetically engineered strain of Serratia marcescens, the present disclosure can further increase the yield of spermidine up to 26.8 g/L.
(7) The present disclosure introduces an exogenous pathway, and realizes a novel synthetic pathway for the synthesis of spermidine in E. coli by incorporating exogenous APADH gene, exogenous CAPADC gene, and exogenous APAUH gene. E. coli produces almost no spermidine without genetic engineering modification, and the yield is extremely low, close to zero. For the traditional synthetic pathway of spermidine in Escherichia coli, spermidine is synthesized from carboxylated-S-adenosylmethionine and putrescine under the action of spermidine synthase. The production efficiency of the carboxylated-S-adenosylmethionine synthesis pathway is low. Compared with the traditional production pathways, the genetically engineered strain of E. coli of the present disclosure can significantly improve the production of spermidine. When only the exogenous pathway is introduced, a concentration of spermidine reaches 6.7 g/L.
(8) The present disclosure eliminates feedback inhibition and repression in the genetically engineered strain of Escherichia coli. The synthesis of arginine is a multi-stage enzyme chain reaction starting from glutamate. It is necessary to remove feedback inhibition of arginine on N-acetylglutamate synthase encoded by argA. ArgR is a repressor protein of the arginine (Arg) synthetic pathway. The present disclosure eliminates the feedback inhibition of arginine and the repression, and removes the repression of the pathway by ArgR. After optimization, the spermidine yield reaches 7.8 g/L.
(9) The present disclosure enhances the expression of key enzymes in the genetically engineered strain of Escherichia coli. Aspartate kinase encoded by the lysC gene and aspartate semialdehyde dehydrogenase encoded by the asd gene are key enzymes that catalyze the synthesis of aspartate semialdehyde from aspartate. N-acetylglutamate synthase, and arginine succinate lyase encoded by argA and argH genes are the first and last enzymes in the arginine synthesis pathway, respectively. The arginine decarboxylase encoded by the speA gene is a key enzyme that catalyzes further synthesis of agmatine from arginine. The expression of the five enzymes can further increase the yield of spermidine to 14.8 g/L.
(10) The present disclosure improves the transport pathway of spermidine in the genetically engineered strain of Escherichia coli. PotABCD and MdtJI proteins are responsible for transporting spermidine into and out of cells, respectively. By knocking out potA and enhancing the expression of mdtJI, the spermidine yield is increased to 18.5 g/L.
(11) The present disclosure weakens the competitive pathways in the spermidine synthesis process in the genetically engineered strain of Escherichia coli. In the synthesis process of spermidine, the intermediate aspartate semialdehyde has multiple competitive pathways for the production of amino acids, such as a lysine pathway and a homoserine pathway. There are also multiple competitive pathways for the glutamic acid intermediate, such as the synthesis of Pro. By weakening the aforesaid pathways, the yield of spermidine can reach 21.3 g/L after knocking out the genes thrA, metL, dapA, and proC.
(12) The present disclosure knocks out related decomposition pathways in the genetically engineered strain of Escherichia coli. E. coli contains enzymes that utilize and degrade the intermediates and products of spermidine synthesis. For example, the arginine N-succinyltransferase encoded by the astA gene degrades arginine into N2-succinylarginine, which is further degraded into L-glutamate and succinic acid through the ast gene series. The yield of spermidine can be increased and reach 24.7 g/L.
(13) The genetically engineered strain of E. coli of the present disclosure can produce a maximum yield of 266 g/L through high-density fermentation.

### Biological Deposit Materials

The Serratia marcescens HBQA7 strain of the present disclosure, taxonomically designated as Serratia marcescens HBQA7, was deposited with China Center for Type Culture Collection (CCTCC) with an accession number CCTCC NO: M 2023184 on Feb. 23, 2023. The deposit address is Wuhan University, Wuhan City, P.R.China.

### BRIEF DESCRIPTION OF THE DRAWINGS

The FIGURE illustrates spermidine biosynthesis pathways in Serratia marcescens and E. coli of the present disclosure.

### DETAILED DESCRIPTIONS OF THE EMBODIMENTS

### 1. Strains and Plasmids Involved in the Present Disclosure

Vector plasmids pRSFDuet-1, pTrc99a, PKD46, pCP20 and T19 purchased from Novagen Company; vector plasmid pMD18-T; and strains Escherichia coli BL21 (DE3), Escherichia coli JM109, Escherichia coli DH5α, Escherichia coli Top10, and Escherichia coli MG1655. pBBRIMCS-2 purchased from MiaoLing Plasmid Platform, and Serratia marcescens HBQA7 (CCTCC NO: M 2023 184) deposited with the laboratory. Illustrative examples of Serratia species include, but are not limited to: Serratia fonticola ATCC 29845; Serratia odorifera ATCC 33077; Serratia plymuthica ATCC 15928; Serratia liquefaciens ATCC 27592; Serratia rubidaea ATCC 19279; Serratia oryzae ATCC 1011; Serratia ureilytica ATCC BAA-2620; Serratia entomophila ATCC 43705; Serratia ficaria ATCC 33105; Serratia marcescens ATCC 13880 and Serratia proteamaculans ATCC 19323 purchased from American Type Culture Collection (ATCC). Serratia symbiotica DSM 23270; Serratia nematodiphila DSM 21420; Serratia quinivorans DSM 4597 and Serratia grimesi DSM 30063 purchased from Deutsche Sammlung von Mikroorganismen und Zellkulturen (DSMZ). The strain Serratia marcescen HBQA7 was deposited with China Center for Type Culture Collection (CCTCC) with an accession number CCTCC NO: M 2023184.

### 2. Fermentation Conditions

For strain yield verification in the present disclosure, a shake-flask fermentation medium is as follows: 10 g glucose, 10 g yeast extract, 20 g NaCl, 50% (v/v) R/2 medium, and 5 mL of a trace metal solution per liter; a R/2 medium composition is as follows: 2 g (NH₄)₂HPO₄, 6.75 g KH₂PO₄, 0.85 g citric acid, 0.7 g MgSO₄·7H₂O per liter and pH 6.86; and composition of the trace metal solution is as follows: 5 M HCl, 10 g FeSO₄·7HO, 2.25 g ZnSO₄·7H₂O, 1g CuSO₄·5H₂O, 0.5 g MnSO₄·5H₂O 0.23 g Na₂B₄O₇·10H₂O, 2 g CaCl₂·2H₂O, and 0.1 g (NH₄)₆Mo₇O₂₄. The shaking flask fermentation was conducted at a temperature of 37° C., with an initial pH of 7.0. A liquid volume was set to 1/5 of a shaking flask volume, a shaking speed was controlled at 200 rpm, and a fermentation duration was 24 hours.

### 3. Method for Determination of Spermidine Content

Preparation of an o-Phthalaldehyde (OPA) derivatization reagent: The OPA reagent was prepared using a Uren method and Karababa. Specifically, 0.20 g OPA was dissolved in 9.0 mL methanol, followed by adding 1.0 mL of 0.40 M borate buffer (pH 9.0) and 160 μL of 2-mercaptoethanol (a reducing reagent). The resulting OPA reagent was stored at 4° C.

Spermidine was derivatized using o-Phthalaldehyde (OPA; Sigma, St. Louis, MO). 50 μL sample and 450 μL water were added to 400 μL methanol to obtain a mixture. 100 μL of the prepared OPA reagent was added to the mixture, the mixture was filtered through a 0.2 μm membrane filter, and 20.0 μL filtrate was immediately injected into an HPLC system.

HPLC detection conditions: Spermidine was detected according to the method in the literature (Qian, Z. G., X. X. Xia, and S. Y. Lee, Metabolic engineering of Escherichia coli for the production of putrescine: a four carbon diamine. Biotechnol Bioeng, 2009. 104 (4): p. 651-662): Samples were separated using a Luna 5-μm C18(2) 100 Å column (250×4.6 mm; Phenomenex) at 25° C. with a flow rate of 0.8 mL/min. A mobile phase consisted of Solvent A (55% methanol in 0.1 M sodium acetate, pH 7.2) and Solvent B (methanol). The following gradient was applied: 1-6 minutes, 100% A; 6-10 minutes, a linear gradient of B from 0% to 30%; 10-15 minutes; a linear gradient of B from 30% to 50%; 15-19 minutes, a linear gradient of B from 50% to 100%; 19-23 minutes, 100% B; 23-25 minutes, a linear gradient of B from 100% to 30%; and 25-28 minutes, a linear gradient of B from 30% to 0% (all in volume %).

### 4. Gene Integration and Expression Scheme

Gene integration and expression were achieved using the principle of homologous recombination. Specifically, a target gene was amplified using primers “KZ-F/R”, and was integrated into a multiple cloning site (MCS) of the T19 vector plasmid via homologous recombination. Using 1000 bp upstream and downstream of the target gene as a template, the left arm (LA) and a right arm (RA) of a homologous arm were amplified and connected to two ends of the target gene on the T19 vector plasmid to construct the homologous arm. Plasmids correctly ligating the left arm and the right arm connected were extracted, the plasmid T19-LA-trc-target gene-RA was used as a template to amplify a target fragment LA-trc-target gene-RA using the corresponding primer. After the PCR product was purified and treated with the restriction enzyme DpnI to eliminate template effect, and the product after enzyme digestion was purified again. The plasmid PKD46, which expressed homologous recombinase, was extracted using a plasmid extraction kit, purified, and then transformed into competent cells of Serratia marcescens HBQA7 to construct Serratia marcescens HBQA7-PKD46. After preparation, the competent cells of Serratia marcescens HBQA7-PKD46 were stored at −80° C. for subsequent use. The purified product was introduced into the above-mentioned competent cells of Serratia marcescens. Positive transformants were screened on resistance plates using a kanamycin resistance marker. In order to verify whether recombination occurred, PCR was performed using the primers “target gene-YZ-F” and “target gene-YZ-R.” When recombination had occurred, a fragment of approximately 1200 bp could be amplified; otherwise, no fragment could be amplified. In addition, Serratia marcescens were verified on ampicillin and kanamycin dual-resistance plates using the corresponding PCR method, confirming that the correct transformants Serratia marcescens HBQA7-PKD46 (kana^{R}) was obtained. Subsequently, kanamycin resistance was eliminated. First, competent cells of Serratia marcescens HBQA7-PKD46 (kana^{R}) were prepared, and the plasmid pCP20 was transformed into the competent cells of Serratia marcescens HBQA7-PKD46 (kana^{R}). Transformants in which the resistance had been eliminated were screened using both non-resistant and resistant plates. Colony PCR was then performed to identify transformants that had integrated the target gene and eliminated resistance.

Using the above gene integration and expression method and corresponding operation steps, mutated genes and substituted promoter sequences were integrated into genomic locations to achieve metabolic engineering modification of the strain.

Restriction digestion reaction system: 10×Q Cut Buffer 5 μL, DNA 20 μL, ddH₂O 33 μL, 1 μL restriction enzyme 1, 1 μL restriction enzyme 2, mixed thoroughly and incubated in a 37° C. water bath for 0.5-1 hour.

Ligation reaction system: 5 μL lightning cloning enzyme; 1-2 μL double-digested plasmid (10-100 ng); 4-5 μL homologous target fragment; ddH₂O to a total volume of 10 μL, mixed thoroughly and incubate in a 50° C. water bath for 30 minutes.

DpnI template effect elimination reaction system (60 μL): 3 μL DpnI, 20 μL purified PCR product, 10×FastDigest Buffer 6 μL, and 31 μL ddH₂O.

PCR reaction system for target gene amplification (50 μL): 1 μL DNA template, 10 μL 5×PrimeSTAR Buffer, 4 μL dNTP Mixture, 1 μL upstream amplification primer, 1 μL downstream amplification primer, 0.5 μL PrimeSTAR HS DNA Polymerase, and 32.5 μL ddH₂O.

PCR amplification program: 30 cycles (98° C. for 10 seconds, 55° C. for 10 seconds, 72° C. at 1 kb/min), 72° C. for 10 minutes, and storage at 4° C.

Colony PCR reaction system: 2×Tag Master Mix 10 μL, Forward primer (a concentration of 10 μM), 1 μL; reverse primer (a concentration of 10 μM), 1 μL; ddH₂O 8 μL, and a small amount of bacterial colony or culture as template.

### EXAMPLE 1

### Introduction of Exogenous Spermidine Synthesis Pathway—Construction of Genetically Engineered Strain of Serratia marcescens

The present disclosure introduces the CAPADH/CAPADC/APAUH pathway derived from different microbial strains into Serratia marcescens HBQA7 for heterologous expression. A schematic diagram of the construction is shown in the figure. First, various carboxyaminopropylagmatine dehydrogenases (CAPADH), carboxyaminopropylagmatine decarboxylases (CAPADC), and aminopropylagmatine ureahydrolases (APAUH) from different microbial sources were expressed in Serratia marcescens via plasmids. An enzyme with highest catalytic efficiency was further selected, and the CAPADH/CAPADC/APAUH pathway was exogenously integrated through homologous recombination. A genomic integration site selected in the present disclosure was located upstream of an argA gene in the Serratia marcescens HBQA7.

The specific procedure process is as follows:

Using amplification primers, CAPADH genes stcapadh, dacapadh, gscapadh and iccapadh were cloned from genomes of Spirochaeta thermophila DSM 6578, Denitrovibrio acetiphilus DSM 12809, Geobacter sulfurreducens KN400 and Ilumatobacter coccineus YM16-304, respectively, and amino acid sequences of the cloned genes were listed in the NCBI database under accession numbers of WP_014623968.1, WP_013011851.1, WP_010943175.1, and WP_015442124.1, respectively; CAPADC genes stcapade, dacapade, gscapade and iccapade were cloned from genomes of Spirochaeta thermophila DSM 6578, Denitrovibrio acetiphilus DSM 12809, Geobacter sulfurreducens KN400 and Ilumatobacter coccineus YM16-304, respectively, and amino acid sequences of the cloned genes were listed in the NCBI database under accession numbers of WP_014623969.1, WP_013011852.1, WP_004513933.1 and WP_015442123.1, respectively; and APAUH genes stapauh, daapauh, Ibapauh and icapauh were cloned from genomes of Spirochaeta thermophila DSM 6578, Denitrovibrio acetiphilus DSM 12809, Geobacter sulfurreducens KN400 and Ilumatobacter coccineus YM16-304, respectively, and amino acid sequences of the cloned genes were listed in the NCBI database under accession numbers of WP_014623970.1, WP_013011853.1, WP_015770028.1 and WP_015442122.1, respectively. The three types of cloned genes were ligated into a same plasmid pBBRIMCS-2 to obtain recombinant Serratia marcescens with enhanced expression of the three genes.

The CAPADH and CAPADC in the aforementioned microorganisms are located adjacent to each other within a gene cluster. Using stcapadh-HindIII-F and stcapade-EcoRV-R as primers, and Spirochaeta thermophila DSM 6578 genome as a template, a target fragment stcapadh-stcapade was amplified; and a first multiple cloning site of the pBBRIMCS-2 plasmid was digested with HindIII and EcoRV. After purification of the digestion product, the digested plasmid was ligated with the amplified target fragment stcapadh-stcapadc, the ligated product pBBRIMCS-2-stcapadh-stcapadc was transformed into competent E. coli JM109 cells. Positive clones were selected on kanamycin-resistant plates and further verified by colony PCR. Using stapauh-EcoRI-F and stapauh-SpeI-R as primers, and Spirochaeta thermophila DSM 6578 genome as a template, a target fragment stapauh was amplified; and a second multiple cloning site of the pBBRIMCS-2-stcapadh-stcapadc was digested with EcoRI and SpeI. After purification of the digestion product, the digested plasmid was ligated with the amplified target fragment stapauh, the ligated product pBBRIMCS-2-stcapadh-stcapadc-stapauh was transformed into competent JM109 cells. Positive clones were selected on kanamycin-resistant plates and further verified by colony PCR. The JM109 strain that was successfully ligated was selected, and the plasmid pBBRIMCS-2-stcapadh-stcapade-stapauh was extracted and transformed into competent cells Serratia marcescens HBQA7 to construct a spermidine synthesis strain Serratia marcescens HBQA7-stcapadh-stcapade-stapauh. The construction methods for other strains were similar to that of the Serratia marcescens HBQA7-stcapadh-stcapadc-stapauh. Specifically, CAPADH, CAPADC, and APAUH from different strains were ligated into the two multiple cloning sites of the plasmid pBBRIMCS-2. The ligation and methods of other genes were similar to the above. The primer sequences used are listed in Table 1.

TABLE 1
Primer sequences (SEQ ID NO: 1-4)

| Primer name | Sequence 5′-3′ | SEQ ID |
|---|---|---|
| stcapadh- | GCACCCCAGGCAAGCTTGATGGGCAC | NO: 1 |
| HinIII-F | GGGTGCTCATCAT | |
| stcapadc- | GGGCTGCAGGAATTCGATATCTTACG | NO: 2 |
| EcoRV-R | AGAGGCGCCGCTT | |
| stapauh- | CGATGATATCGAATTCATGCGCTATC | NO: 3 |
| EcoRI-F | CCCACTTCCT | |
| stapauh- | AGTTCTAGAACTAGTCTACCGCCGGG | NO: 4 |
| SpeI-R | GTAGATGCC | |

Recombinant Serratia marcescens was inoculated into a shake-flask fermentation medium (10 g/L peptone, 5 g/L yeast extract, and 10 g/L NaCl) at a volume ratio of 2%. When the cells reached an OD₆₀₀ of 0.6-0.8, IPTG was added to a final concentration of 0.4 mM to induce gene expression at 20° C. for 12 hours. Upon completion of induction, a supernatant was collected with centrifugation at 8000 rpm at 4° C. for 20 minutes. A spermidine concentration in the supernatant was determined by high-performance liquid chromatography (HPLC), as shown in Table 2.

TABLE 2

| Recombinant strains | Spermidine (g/L) |
|---|---|
| Serratia marcescens-stcapadh-stcapadc-stapauh | 5.9 |
| Serratia marcescens-iccapadh-iccapadc-icapauh | 1.8 |
| Serratia marcescens-dacapadh-dacapadc-daapauh | 4.6 |
| Serratia marcescens-gscapadh-gscapadc-lbapauh | 2.4 |
| Serratia marcescens-stcapadh-gscapadc-stapauh | 4.1 |
| Serratia marcescens-gscapadh-stcapadc-stapauh | 2.7 |
| Serratia marcescens-stcapadh-dacapadc-stapauh | 3.3 |
| Serratia marcescens-dacapadh-stcapadc-stapauh | 2.8 |

Overexpressing key genes involved in spermidine synthesis via expression vectors imposes a metabolic burden on the bacterial cells, thereby affecting their growth. The expression vectors suffered from the structural instability and easy loss during proliferation of the bacterial cells, as well as fluctuations in plasmid copy number, in addition, the use of antibiotics inhibited cell growth and posed environmental contamination risks, while IPTG induction increased production costs, thus limiting the scalability of production. Therefore, the recombinant strain Serratia marcescens-stcapadh-stcapadc-stapauh, which demonstrated the best effect of spermidine synthesis, was selected. An exogenous gene expression fragment was constructed by fusing a promoter J23119 in the Serratia marcescens and a ribosome binding site (RBS) B0034 with the gene stcapadh-stcapadc-stapauh. According to the aforesaid section “4. Gene integration and expression scheme”, the exogenous gene expression fragment was integrated into a knockout strain Serratia marcescens HBQA7 to construct a strain Serratia marcescens-stcapadh-stcapadc-stapauh, designated as a strain S1.

Shake flask culture was conducted to verify spermidine yield of the strain S1. A concentration of spermidine in the supernatant in the shake flask was determined to be 5.9 g/L.

### EXAMPLE 2

### Relief of Feedback Inhibition of Key Enzymes in the Genetically Engineered Strain of Serratia marcescens Pathway and Knockout of Repressor Protein

In the synthesis of key intermediates in aspartate semialdehyde and arginine, a repressor protein inhibits the synthesis of both intermediates and final products. In the synthesis of key intermediates in aspartate semialdehyde and arginine, a repressor protein inhibited the synthesis of both intermediates and final products. An argR gene (with an amino acid sequence available under an NCBI accession number WP_004933572) encoded a DNA-binding dual transcriptional regulator that repressed the expression of an arg gene series, and inhibited the excessive synthesis of key intermediates such as ornithine and arginine. Therefore, in order to ensure stable and high-rate synthesis of the intermediates, the gene encoding the repressor protein was deleted to increase a metabolic flux.

In the metabolic pathway of arginine synthesis, N-acetylglutamate synthase (amino acid sequence available under an NCBI accession number ALE97678.1) encoded by the argA gene was subjected to feedback inhibition by its product arginine. In order to improve the enzyme activity, base mutation of the gene encoding the enzyme was further conducted on the basis of knocking out the argR gene to construct a negative feedback mutant, thereby releasing feedback inhibition and obtaining a resulting engineered strain Serratia marcescens argA^{fbr} ΔArgR, designated as a strain S2.

The specific procedure process is as follows:

### (1) Knockout of the Repressor Protein ArgR

Primer design is listed in Table 3 below:

TABLE 3
Primer sequences (SEQ ID NO: 5-14)

| Primer name | Primer sequence 5′-3′ | SEQ ID |
|---|---|---|
| ApaI-LA-argR | CGCGCGGATCTTCCAGAGATTGGG | NO: 5 |
| | CCCACCTGCCAGAGTGGCCGCCG | |
| argR-LA- | AAGCTTGACGTCCAGGTGCCTCTT | NO: 6 |
| EcoRI | AAGTTTGCACCGTGGAATGTT | |
| KpnI-RA-argR | CACGCTAGCGGATCCGAGCTCGGT | NO: 7 |
| | ACCAACCTGGCGCTAACAAAGT | |
| argR-RA-SalI | CGTGCGGACGGCAAGCTGCTACAG | NO: 8 |
| | CTGGATCACGCGGTAGGCG | |
| argR-LA-YZ-F | tgaattagaactcggtacgcgcgg | NO: 9 |
| | a | |
| argR-LA-YZ-R | tagagaataggaacttcgaactgc | NO: 10 |
| | a | |
| argR-RA-YZ-F | aacttcgaagcagctccagcctac | NO: 11 |
| | a | |
| argR-RA-YZ-R | atgattacgccaagtttgcacgcc | NO: 12 |
| | t | |
| argR-YZ-F | TTCAGTGCCGGCGTTCTGAATA | NO: 13 |
| argR-YZ-R | Cgccctgagtgcttgcggcagcgt | NO: 14 |
| | g | |

The plasmid PKD46, which expressed homologous recombinase, was extracted using a plasmid extraction kit, purified, and then transformed into competent cells of S1 to construct S1-PKD46. After preparation, the competent cells of S1-PKD46 were stored at −80° C. for subsequent use. A plasmid T19, serving as a template plasmid for homologous recombination fragment construction, was extracted using a plasmid extraction kit and purified with a gel extraction kit. Primer pairs ApaI-LA-argR and argR-LA-EcoRI, as well as KpnI-RA-argR and argR-RA-SalI were designed to amplify the corresponding homologous arm left arm (LA) and right arm (RA) fragments, which were purified and recovered using a gel extraction kit. The plasmid T19 was digested with ApaI and EcoRI, and the digestion results were detected by agarose gel electrophoresis. A size of the digested fragment was 5041bp, and the digestion product was purified and recovered. The product after double-enzyme digestion of the T19 vector was ligated with the left arm (LA) fragment. The ligated product was transformed into competent E. coli JM109 cells, and plated on a kanamycin-resistant plate. Positive clones are screened and verified by colony PCR using primers LA-YZ-F and LA-YZ-R, producing a band with a size of approximately 1100 bp. Verified clones were sent for sequencing to further confirm successful ligation of the fragments. Subsequently, the plasmid containing the LA fragment was digested with KpnI and SalI. The RA fragment was ligated using the same method, and the ligated plasmid was verified. The plasmid containing both the LA and RA fragments was extracted and used as a template for PCR with primers ApaI-LA-argR and argR-RA-SalI to obtain a target fragment argR-LA-FRT-Kan-FRT-RA. Agarose gel electrophoresis confirmed an expected size of the PCR product was approximately 3324 bp. After the PCR product was purified and treated with the restriction enzyme DpnI to eliminate template effect. After digestion with the restriction enzyme DpnI, the PCR product of the target fragment argR-LA-FRT-Kan-FRT-RA was further purified and then transformed into competent cells Serratia marcescens HBQA-PKD46. Positive transformants were screened on resistance plates using a kanamycin resistance marker. In order to recombination in the screened positive transformants had occurred, primers argR-YZ-F and argR-YZ-R were used as templates. When a fragment of approximately 1100 bp was amplified, it was confirmed that recombination had occurred. Otherwise, when no fragment was amplified, a false-positive clone was confirmed. When verifying whether recombination had occurred by PCR amplification, the corresponding PCR verification bacteria were spotted on ampicillin and kanamycin dual-resistance plates. A correct transformant S1-PKD46 (kana^{R}) that was confirmed to have undergone recombination was subjected to elimination by a kanamycin resistance marker. First, competent cells of S1-PKD46 (kana^{R}) were prepared, and the extracted plasmid pCP20 was transformed into the competent cells of S1-PKD46 (kana^{R}). Transformants in which the resistance had been eliminated were screened using both non-resistant and resistant plates. Colony PCR was then performed, and a transformant S1-ΔargR with knockout of the argR gene and eliminated resistance were screened.

(2) Construction of argA^{fbr} Mutant

The argA gene was subjected to feedback inhibition by arginine, the target product. In the present disclosure, a point mutation was introduced at a 40th nucleotide of the argA gene, changing a cytosine (C) to thymine (T), and the encoded histidine was converted into tyrosine, thereby significantly increasing arginine production. Therefore, the mutant gene at the site was integrated into a plasmid pRSFDuet-1.

The specific procedure process is as follows:
Primer design is listed in Table 4 below:

TABLE 4
Primer sequences (SEQ ID NO: 15-18)

| | Primer sequence | |
|---|---|---|
| Primer name | 5′-3′ | SEQ ID |
| F-argA-amplification | GTGAAGGAACGTAGTACAG | NO: 15 |
| primer | AGCT | |
| R-argA-amplification | TTACAGATCCGCCAGCAGA | NO: 16 |
| primer | | |
| argA-TB-F | CAAGGATTCCGTTACTCAGTC | NO: 17 |
| argA-TB-R | GACGGAATCCTTGCACCAGCT | NO: 18 |

First, a plasmid pRSFDuet-1 (high-copy) was extracted using a plasmid extraction kit, and was subjected to double digestion with BamHI and HindIII. A restriction digestion system (50 μL) was as follows: 5μL 10×Q Cut Buffer, plasmid 20 μL, 1 μL BamHI, 1 μL HindIII, and 33 μL ddH2O. After digestion, the product was purified. Next, a Serratia marcescens HBQA genome was extracted and used as a template to amplify the argA gene fragment. After verification, the PCR product was ligated into the digested plasmid pRSFDuet-1. Competent E. coli JM109 cells were prepared using a CaCl₂ method. The plasmid pRSFDuet-1 carrying the argA fragment was then transformed into the competent cells. Positive transformants were selected and identified by colony PCR as the successfully transformed strain E. coli JM109-argA. A plasmid pRSFDuet1-argA was extracted from E. coli JM109-argA and used as a template for site-directed mutagenesis of argA. Mutant genes were designed and extracted using a Fast Mutangenesis System kit. The extracted plasmid pRSFDuet1-argA was used as a template, and an argA^{fbr} fragment was amplified using the argA mutant primers argA-TB-F/R. A PCR amplification system (50 μL) was as follows: 3 μL plasmid template, 1 μL argA-TB-F, 1 μL argA-TB-R, 25 μL 2×TransStart®FastPfu Fly PCR SuperMix, and 20 μL ddH₂O. PCR amplification program was as follows: 94° C. for 2-5 minutes, 20-25 cycles (94° C. for 20 seconds, 55° C. for 20 seconds, 72° C. at 2-6 kb/min), 72° C. for 10 minutes, and storage at 4° C. The resulting PCR product was digested with 1-3 μL of DMT enzyme, mixed well, and incubated at 37° C. for 1 hour. The DMT digestion product was then purified using a PCR product purification kit. 2-5 μL was taken and added to 50 μL of competent JM109 cells for transformation. Positive transformants were selected and identified by colony PCR, and then sequenced by a sequencing company to identify the correct transformant, JM109-argA^{fbr}.

The plasmid pRSFDuet1-argA^{fbr} was extracted from the transformant JM109-argA^{fbr}. According to the aforesaid section “4. Gene integration and expression scheme”, the argA^{fbr} fragment was integrated into the knockout strain S1-ΔargR to construct the recombinant strain S1-argA^{fbr} ΔArgR, designated as strain S2, for subsequent studies.

Shake flask culture was conducted to verify arginine production of the strain S2. A concentration of arginine in the supernatant in the shake flask was determined to be 8.6 g/L.

### EXAMPLE 3

### Enhanced Expression of Key Enzymes in the Genetically Engineered Strain of Serratia marcescens Pathway

In the present disclosure, expression of key enzymes involved in the arginine was enhanced by replacing a native promoter with a strong trc promoter, thereby increasing metabolic flux toward arginine synthesis. The trc promoter SEQ ID NO:187: 5′-TTGACAATTAATCATCCGGCTCGTATAATG-3′) was derived from a plasmid pTrc99a. The aspartate kinase encoded by the lysC gene catalyzed the phosphorylation of aspartate. The aspartate semialdehyde dehydrogenase encoded by asd gene catalyzed the phosphorylated aspartate to generate aspartate semialdehyde. In order to enhance the enzyme activity and flux of the metabolic pathway, the native promoter of the gene was replaced with the strong trc promoter to promote higher gene expression and increased production of the desired product.

Using the strain S2 as a parental strain, the expression of the following key enzymes in the synthetic pathway was enhanced: lysC (with an amino acid sequence available under an NCBI accession number WP_004936686.1), asd (with an amino acid sequence available under an NCBI accession number TEW88852.1), argA (with an amino acid sequence available under an NCBI accession number ALE97678.1), argH (with an amino acid sequence available under an NCBI accession number TEW89265.1) and speA (with an amino acid sequence available under an NCBI accession number TEW85704.1). By replacing the native promoter of the gene with the strong trc promoter, the engineered strain, designated as a strain S2-P_{trc}lysC-P_{trc}asd-P_{trc}argA-P_{trc}argH-P_{trc}speA, was obtained.

The specific procedure process is as follows:

Based on the lysC sequence of Serratia marcescens HBQA in NCBI, primers were designed using Primer Premier 5 (see Table 5). A target gene fragment, lysC, was amplified by PCR using Serratia marcescens HBQA genome as a template and lysC-KZ-F and lysC-KZ-R as primers, with EcoRI and HindIII restriction sites added upstream and downstream, respectively, The PCR fragment was purified. The plasmid pTrc99a was digested with EcoRI and HindIII, purified, and ligated with the purified lysC fragment for transformation into competent E. coli JM109 cells. Positive transformants were screened and verified by colony PCR using verification primers lysC-99a-YZ-F/R, and further confirmed by sequencing. The successfully transformed plasmid pTrc-lysC was extracted and used as a template. The target fragment trc-lysC was amplified using BamHI-trc-lysC-F and trc-lysC-SacI-R as primers. The target fragment was verified to be approximately 1398 bp by agarose gel electrophoresis and purified. The T19 vector plasmid was digested with BamHI and SacI, purified, and ligated with the trc-lysC fragment for transformation. The ligated product was verified by colony PCR with primers trc-lysC-T19-YZF/YZR, followed by sequencing, and the correct positive transformant T19-trc-lysC was selected. Using the lysC genome in Serratia marcescens HBQA as a template, the left arm (LA) fragment was amplified using lysC-LA-ApaI and EcoRI-lysC-LA as primers, and the right arm (RA) fragment was amplified using lysC-RA-KpnI and SalI-lysC-RA as primers. After purification, the plasmid was ligated to the left arm and the right arm of the double-digested and purified plasmid T19-trc-lysC, respectively. The ligated product was verified by colony PCR with verification primers lysC-LA-YZ-F and lysC-LA-YA-R, as well as lysC-RA-YZ-F and lysC-RA-YA-R, followed by sequencing to identify to the plasmid T19-LA-trc-lysC-RA with the left arm and the right arm correctly ligated. The recombinant plasmid T19-LA-trc-lysC-RA, with the left arm and the right arm correctly ligated, was extracted and used as the template to amplify the target fragment LA-trc-lysC-RA using lysC-LA-ApaI and SalI-lysC-RA as primers. The fragment was approximately 3700 bp in size. The PCR product was purified and treated with the restriction enzyme DpnI to remove template effect. The digested product was further purified and then transformed into S2-PKD46 (the homologous recombinase expression plasmid PKD46 is extracted using a plasmid extraction kit, purified, and transformed into competent S2 cells to construct S2-PKD46) competent cells. Positive transformants were screened on resistance plates using a kanamycin resistance marker. When recombination had occurred, a fragment of approximately 1000 bp could be amplified using the primers lysC-YZ-F and lysC-YZ-R; otherwise, no fragment could be amplified. In addition, the corresponding PCR verification bacteria were spotted on ampicillin and kanamycin dual-resistance plates. The correct transformant S2-PKD46 (kana^{R}) was identified. Kanamycin resistance was eliminated. First, competent cells of S2-PKD46 (kana^{R}) were prepared, and the plasmid pCP20 was transformed into the competent cells of S2-PKD46 (kana^{R}). Transformants in which the resistance had been eliminated were screened using both non-resistant and resistant plates. Colony PCR was then performed to identify the transformant S2-P_{trc}lysC that replaced the lysC gene promoter and eliminated resistance.

Following the same strategy, the promoters of the genes asd, argA, argH and speA were replaced in succession. A strain S2-P_{trc}lysC-P_{trc}asd-P_{trc}argA-P_{trc}argH-P_{trc}speA was constructed, which was designated as S3. Primers used are shown in Table 5 below.

TABLE 5
Primer sequences (SEQ ID NO: 19-108)

| Primer name | Primer sequence 5′-3′ | SEQ ID |
|---|---|---|
| LysC-KZ-F | TGGAATTCATGAACCAAGCAGCACC | NO: 19 |
| LysC-KZ-R | GCCAAGCTTTTATTCGAACAGGTTGTGATGCAG | NO: 20 |
| lysC-99a-YZ-F | TCACTGCATAATTCGTGTCGCTCAA | NO: 21 |
| lysC-99a-YZ-R | CGCTACTGCCGCCAGGCAAATTCTG | NO: 22 |
| BamHI-trc- | AGCCTACACGCTAGCGGATCCTTGACAATTAATCATC | NO: 23 |
| lysC-F | CGGC | |
| trc-lysC- | AGTATCTCAGGTACCGAGCTCTTATTCGAACAGGTTG | NO: 24 |
| SacI-R | TGATGCAG | |
| trc-lysC-T19-YZF | tcttctaataaggggatcttgaagt | NO: 25 |
| trc-lysC-T19-YZR | cgctgttgcggatcatctccagcgt | NO: 26 |
| lysC-LA-ApaI | CGCGCGGATCTTCCAGAGATTGGGCCCGGCGCGCTG | NO: 27 |
| | GCTGCGG | |
| EcoRI-lysC-LA | AAGCTTGACGTCCAGGTGCCTCTTAAGCTAGAAACCT | NO: 28 |
| | CGTGTCAGG | |
| lysC-RA-KpnI | CACGCTAGCGGATCCGAGCTCGGTACCGTCATTATCC | NO: 29 |
| | CTGATTGCGGGC | |
| SalI-lysC-RA | CGTGCGGACGGCAAGCTGCTACAGCTGGCCAGACCG | NO: 30 |
| | AGAACAGCG | |
| lysC-LA-YZ-F | tgaattagaactcggtacgcgcgga | NO: 31 |
| lysC-LA-YZ-R | tagagaataggaacttcgaactgca | NO: 32 |
| lysC-RA-YZ-F | aacttcgaagcagctccagcctaca | NO: 33 |
| lysC-RA-YZ-R | atgattacgccaagtttgcacgcct | NO: 34 |
| LysC-YZ-F | TGCAGCGCAGCGGCTATC | NO: 35 |
| LysC-YZ-R | Cgccctgagtgcttgcggcagcgtg | NO: 36 |
| asd-KZ-F | AGGAAACAGACCATGGAATTCATGAAAAACGTTGGT | NO: 37 |
| | TTCATCGGTT | |
| asd-KZ-R | TCCGCCAAAACAGCCAAGCTTTTACAGCAGGATGCG | NO: 38 |
| | CAG | |
| asd-99a-YZ-F | TCACTGCATAATTCGTGTCGCTCAA | NO: 39 |
| asd-99a-YZ-R | CGCTACTGCCGCCAGGCAAATTCTG | NO: 40 |
| BamHI-trc-asd-F | AGCCTACACGCTAGCGGATCCTTGACAATTAATCATC | NO: 41 |
| | CGGC | |
| trc-asd-SacI-R | ATCAGTATCTCAGGTACCGAGCTCTTACAGCAGGATG | NO: 42 |
| | CGCAG | |
| trc-asd-T19-YZF | tcttctaataaggggatcttgaagt | NO: 43 |
| trc-asd-T19-YZR | cgctgttgcggatcatctccagcgt | NO: 44 |
| asd-LA-Apal | CGCGCGGATCTTCCAGAGATTGGGCCCCGAGGAGCG | NO: 45 |
| | TCTGAAGCAAC | |
| EcoRI-asd-LA | AAGCTTGACGTCCAGGTGCCTCTTAAGAATATCTGTG | NO: 46 |
| | TCCCGCCCG | |
| asd-RA-KpnI | CACGCTAGCGGATCCGAGCTCGGTACCACCCGCACTT | NO: 47 |
| | CCAGGGG | |
| SalI-asd-RA | CGTGCGGACGGCAAGCTGCTACAGCTGTGGCCGGCC | NO: 48 |
| | TGCAC | |
| asd-LA-YZ-F | tgaattagaactcggtacgcgcgga | NO: 49 |
| asd-LA-YZ-R | tagagaataggaacttcgaactgca | NO: 50 |
| asd-RA-YZ-F | aacttcgaagcagctccagcctaca | NO: 51 |
| asd-RA-YZ-R | atgattacgccaagtttgcacgcct | NO: 52 |
| asd-YZ-F | GCCATGCAGCGCACC | NO: 53 |
| asd-YZ-R | Cgccctgagtgcttgcggcagcgtg | NO: 54 |
| argA-KZ-F | AGGAAACAGACCATGGAATTCGTGAAGGAACGTAGT | NO: 55 |
| | ACAGA | |
| argA-KZ-R | TCCGCCAAAACAGCCAAGCTTTTACAGATCGGCCAG | NO: 56 |
| | CAGGATT | |
| argA-99a-YZ-F | TCACTGCATAATTCGTGTCGCTCAA | NO: 57 |
| argA-99a-YZ-R | CGCTACTGCCGCCAGGCAAATTCTG | NO: 58 |
| BamHI-trc-argA-F | AGCCTACACGCTAGCGGATCCTTGACAATTAATCATC | NO: 59 |
| | CGGC | |
| trc-argA-SacI-R | ATCAGTATCTCAGGTACCGAGCTCTTACAGATCGGCC | NO: 60 |
| | AGCAGGATT | |
| trc-argA-T19-YZF | tcttctaataaggggatcttgaagt | NO: 61 |
| trc-argA-T19-YZR | cgctgttgcggatcatctccagcgt | NO: 62 |
| argA-LA-ApaI | CGCGCGGATCTTCCAGAGATTGGGCCCGCCTTGGCGA | NO: 63 |
| | CGCGCAC | |
| EcoRI-argA-LA | AAGCTTGACGTCCAGGTGCCTCTTAAGGGCCAAACC | NO: 64 |
| | TCTTTGCATATT | |
| argA-RA-KpnI | CACGCTAGCGGATCCGAGCTCGGTACCTCTCCGCGGC | NO: 65 |
| | GCGGC | |
| SalI-argA-RA | CGTGCGGACGGCAAGCTGCTACAGCTGGTGATTTTCC | NO: 66 |
| | TCGGCGATCG | |
| argA-LA-YZ-F | tgaattagaactcggtacgcgcgga | NO: 67 |
| argA-LA-YZ-R | tagagaataggaacttcgaactgca | NO: 68 |
| argA-RA-YZ-F | aacttcgaagcagctccagcctaca | NO: 69 |
| argA-RA-YZ-R | atgattacgccaagtttgcacgcct | NO: 70 |
| argA-YZ-F | GCCTTTGGTGGACAATGCGAA | NO: 71 |
| argA-YZ-R | Cgccctgagtgcttgcggcagcgtg | NO: 72 |
| ArgH-KZ-F | AGGAAACAGACCATGGAATTCATGGCACTTTGGGGC | NO: 73 |
| | GG | |
| ArgH-KZ-R | TCCGCCAAAACAGCCAAGCTTTCAAGCCAAACGCTG | NO: 74 |
| | T | |
| argH-99a-YZ-F | TCACTGCATAATTCGTGTCGCTCAA | NO: 75 |
| argH-99a-YZ-R | CGCTACTGCCGCCAGGCAAATTCTG | NO: 76 |
| BamHI-trc-argH-F | AGCCTACACGCTAGCGGATCCTTGACAATTAATCATC | NO: 77 |
| | CGGC | |
| trc-argH-SacI-R | ATCAGTATCTCAGGTACCGAGCTCTCAAGCCAAACGC | NO: 78 |
| | TGT | |
| trc-argH-T19-YZF | tcttctaataaggggatcttgaagt | NO: 79 |
| trc-argH-T19-YZR | cgctgttgcggatcatctccagcgt | NO: 80 |
| argH-LA-ApaI | CGCGCGGATCTTCCAGAGATTGGGCCCAATGGCGCG | NO: 81 |
| | GCCGATC | |
| EcoRI-argH-LA | AAGCTTGACGTCCAGGTGCCTCTTAAGACCTTTACTC | NO: 82 |
| | CTGAATTTTT | |
| argH-RA-KpnI | CACGCTAGCGGATCCGAGCTCGGTACCCGACGACAA | NO: 83 |
| | GGGCGG | |
| SalI-argH-RA | CGTGCGGACGGCAAGCTGCTACAGCTGATGCCGGCA | NO: 84 |
| | GCTTCC | |
| argH-LA-YZ-F | tgaattagaactcggtacgcgcgga | NO: 85 |
| argH-LA-YZ-R | tagagaataggaacttcgaactgca | NO: 86 |
| argH-RA-YZ-F | aacttcgaagcagctccagcctaca | NO: 87 |
| argH-RA-YZ-R | atgattacgccaagtttgcacgcct | NO: 88 |
| ArgH-YZ-F | AGCGCAGCGATCTGGAAG | NO: 89 |
| ArgH-YZ-R | Cgccctgagtgcttgcggcagcgtg | NO: 90 |
| speA-KZ-F | AGGAAACAGACCATGGAATTCATGTCTGATGATCTGT | NO: 91 |
| | TGA | |
| speA-KZ-R | TCCGCCAAAACAGCCAAGCTTTTACTCGTCTTCCAGA | NO: 92 |
| | TACGTGT | |
| speA-99a-YZ-F | TCACTGCATAATTCGTGTCGCTCAA | NO: 93 |
| speA-99a-YZ-R | CGCTACTGCCGCCAGGCAAATTCTG | NO: 94 |
| BamHI-trc-speA-F | AGCCTACACGCTAGCGGATCCTTGACAATTAATCATC | NO: 95 |
| | CGGC | |
| trc-speA-SacI-R | ATCAGTATCTCAGGTACCGAGCTCTTACTCGTCTTCCA | NO: 96 |
| | GATACGTGT | |
| trc-speA-T19-YZF | tcttctaataaggggatcttgaagt | NO: 97 |
| trc-speA-T19-YZR | cgctgttgcggatcatctccagcgt | NO: 98 |
| speA-LA-ApaI | CGCGCGGATCTTCCAGAGATTGGGCCCATGGCGTCGA | NO: 99 |
| | GGACG | |
| EcoRI-speA-LA | AAGCTTGACGTCCAGGTGCCTCTTAAGGGTTCGCCTC | NO: 100 |
| | ACATTCCG | |
| speA-RA-KpnI | CACGCTAGCGGATCCGAGCTCGGTACCCCGCCCTCTT | NO: 101 |
| | CAGGGG | |
| SalI-spe A-RA | CGTGCGGACGGCAAGCTGCTACAGCTGCCAACGATG | NO: 102 |
| | TTCAGCGACT | |
| speA-LA-YZ-F | tgaattagaactcggtacgcgcgga | NO: 103 |
| speA-LA-YZ-R | tagagaataggaacttcgaactgca | NO: 104 |
| speA-RA-YZ-F | aacttcgaagcagctccagcctaca | NO: 105 |
| speA-RA-YZ-R | atgattacgccaagtttgcacgcct | NO: 106 |
| speA-YZ-F | ATCGACCCAGGCGCTG | NO: 107 |
| speA-YZ-R | Cgccctgagtgcttgcggcagcgtg | NO: 108 |

Shake flask culture was conducted to verify spermidine yield of the strain S3. A concentration of spermidine in the supernatant in the shake flask was determined to be 16.3 g/L.

### EXAMPLE 4

### Enhanced Transport Pathway in Genetically Engineered Strain of Serratia marcescens

PotABCD is a transporter protein complex in Serratia marcescens responsible for the uptake of spermidine into the cell; therefore, it was knocked out. The PotABCD protein complex is composed of four subunits encoded by gene clusters potA, potB, potC, and potD. Specifically, the potA encodes an ATP-binding subunit, which provides the necessary energy for transport; the potB encodes an ABC transporter membrane subunit PotB; the potC encodes an ABC transporter membrane subunit PotC; and the potD encodes an ABC transporter periplasmic binding protein. Proper expression of all four genes in an operon is essential for functions of the operon. The loss of any single gene will inevitably result in functional inactivation. Therefore, in the present disclosure, the ATP-binding subunit gene potA (with an amino acid sequence available under an NCBI accession number TEW94582.1) was deleted to weaken the capability of transporter proteins to transport spermidine. A strain with an improved transport pathway was constructed based on the strain S3. The PotA was knocked out using the Red homologous recombination method described in Example 2, and the resulting strain was designated as S3-ΔPotA. MdtJI (with an amino acid sequence available under an NCBI accession number TEW81964.1 and TEW81965.1) is a protein in Serratia marcescens responsible for exporting spermidine from the cells. It is encoded by a gene cluster consisting of mdtJ and mdtI. Therefore, its expression was enhanced using a strong trc promoter. Following the method described in Example 2, the promoter was replaced to obtain a strain S3-ΔPotA-P_{trc}MdtJI with enhanced transport pathway, which was designated as a strain S4. Primers used are shown in Table 6 below.

TABLE 6
Primer sequences (SEQ ID NO: 109-136)

| Primer name | Sequence 5′-3′ | SEQ |
|---|---|---|
| ApaI-LA-potA | CGCGCGGATCTTCCAGAGATTGGGCCCGGCTTTCA | NO: 109 |
| | TCCGCCGGAACT | |
| potA-LA-EcoRI | AAGCTTGACGTCCAGGTGCCTCTTAAGGTAAACGC | NO: 110 |
| | AACGGATGGCTT | |
| KpnI-RA-potA | CACGCTAGCGGATCCGAGCTCGGTACCGTTCCAGA | NO: 111 |
| | ATGTAGTGATTG | |
| potA-RA-SalI | CGTGCGGACGGCAAGCTGCTACAGCTGTGTCGTTG | NO: 112 |
| | TTCATCAGCAGG | |
| LA-YZ-F | tgaattagaactcggtacgcgcgga | NO: 113 |
| LA-YZ-R | tagagaataggaacttcgaactgca | NO: 114 |
| RA-YZ-F | aacttcgaagcagctccagcctaca | NO: 115 |
| RA-YZ-R | atgattacgccaagtttgcacgcct | NO: 116 |
| potA-YZ-F | CGGTCGAAATCACGGATGATGTAGT | NO: 117 |
| potA-YZ-R | Cgccctgagtgcttgcggcagcgtg | NO: 118 |
| EcoRI-mdtJI-KZ-F | AGGAAACAGACCATGGAATTCATGATTTATTGGATC | NO: 119 |
| | TTTTTAGGTT | |
| HindIII-mdtJI-KZ-R | TCCGCCAAAACAGCCAAGCTTTCAAACTGTTGCAT | NO: 120 |
| | GGTTGT | |
| mdtJI-99a-YZ-F | TCACTGCATAATTCGTGTCGCTCAA | NO: 121 |
| mdtJI-99a-YZ-R | CGCTACTGCCGCCAGGCAAATTCTG | NO: 122 |
| BamHI-trc-mdtJI-F | AGCCTACACGCTAGCGGATCCTTGACAATTAATCAT | NO: 123 |
| | CCGGC | |
| trc-mdtJI-SacI-R | AGTATCTCAGGTACCGAGCTCTCAAACTGTTGCAT | NO: 124 |
| | GGTTGT | |
| trc-mdtJI-T19-YZF | tcttctaataaggggatcttgaagt | NO: 125 |
| trc-mdtJI-T19-YZR | cgctgttgoggatcatctccagcgt | NO: 126 |
| mdtJI-LA-Apal | CGCGCGGATCTTCCAGAGATTGGGCCCGGAGGCG | NO: 127 |
| | ATCATCGGCG | |
| EcoRI-mdtJI-LA | AAGCTTGACGTCCAGGTGCCTCTTAAGAATTTTTC | NO: 128 |
| | TCCCAAACG | |
| mdtJI-RA-KpnI | CACGCTAGCGGATCCGAGCTCGGTACCTAGCGGGC | NO: 129 |
| | AATAAAAAACC | |
| SalI-mdtJI-RA | CGTGCGGACGGCAAGCTGCTACAGCTGTCTTCCGC | NO: 130 |
| | CACTTCGCTG | |
| mdtJI-LA-YZ-F | tgaattagaactcggtacgcgcgga | NO: 131 |
| mdtJI-LA-YZ-R | tagagaataggaacttcgaactgca | NO: 132 |
| mdtJI-RA-YZ-F | aacttcgaagcagctccagcctaca | NO: 133 |
| mdtJI-RA-YZ-R | atgattacgccaagtttgcacgcct | NO: 134 |
| MdtJI-YZ-F | GCGGTGCCGCTGGCGAAAG | NO: 135 |
| MdtJI-YZ-R | Cgccctgagtgcttgcggcagcgtg | NO: 136- |

Shake flask culture was conducted to verify spermidine yield of the strain S4. A concentration of spermidine in the supernatant in the shake flask was determined to be 21.4 g/L.

### EXAMPLE 5

### Weakening Competitive Pathways in Genetically Engineered Strain of Serratia marcescens

(1) Homoserine Competitive Pathway:

An intermediate substance aspartate semialdehyde was further converted into homoserine by homoserine synthases encoded by thrA (with an amino acid sequence available under an NCBI accession number TEW91726.1) and metL (with an amino acid sequence available under an NCBI accession number ALE98426.1) genes. In order to eliminate the diversion of the competitive metabolic pathway, the thrA and metL genes were deleted based on the strain S4 using the Red homologous recombination method described in Example 2. A resulting strain S4-ΔthrAΔmetL was constructed, which was designated as a strain S5-1.

(2) Lys Competitive Pathways:

The dihydrodipicolinate synthase encoded by a dapA(with an amino acid sequence available under an NCBI accession number TEW94959.1) gene converted the intermediate aspartate semialdehyde into dihydrodipicolinate. To mitigate the impact of this branching pathway and redirect more aspartate toward the metabolic pathway of aspartate semialdehyde, a strain S4-ΔthrAΔmetLΔdapA was constructed by knocking out the dapA gene based on the S4-ΔthrAΔmetL using the Red homologous recombination method described in Example 1. The resulting strain was designated as a S5-2.

(3) Glutamate-derived competitive pathways: Glutamate kinase encoded by proB and glutamate semialdehyde dehydrogenase encoded by proA, glutamate were converted to form glutamate semialdehyde. The glutamate semialdehyde was spontaneously converted to form pyrroline-5-carboxylate, which was then then reduced to proline (pro) by pyrroline-5-carboxylate reductase encoded by proC (with an amino acid sequence available under an NCBI accession number ALE97888.1). Therefore, to block the metabolic flux in the competitive pathways, the proC gene was deleted to weaken the pathway. A strain S4-ΔthrAΔmetLΔdapAΔproC was constructed based on the strain S4-ΔthrAΔmetLΔdapA using the Red homologous recombination method described in Example 2 to delete the competitive pathway gene proC. The resulting strain was designated as a S5-3.

Primers used are shown in Table 7 below.

TABLE 7
Primer sequences (SEQ ID NO: 137-176)

| Primer name | Sequence 5′-3′ | SEQ |
|---|---|---|
| ApaI-LA-thrA | CGCGCGGATCTTCCAGAGATTGGGCCCAAAATG | NO: 137 |
| | TCGGCGCGGC | |
| thrA-LA-EcoRI | AAGCTTGACGTCCAGGTGCCTCTTAAGGTGTGA | NO: 138 |
| | TCTCTCCGAATTTAA | |
| KpnI-RA-thrA | CACGCTAGCGGATCCGAGCTCGGTACCTATGGTT | NO: 139 |
| | AAGGTGTATGCACC | |
| thr A-RA-SalI | CGTGCGGACGGCAAGCTGCTACAGCTGCCTTGT | NO: 140 |
| | TTGATCGCCTGCG | |
| LA-YZ-F | tgaattagaactcggtacgcgcgga | NO: 141 |
| LA-YZ-R | tagagaataggaacttcgaactgca | NO: 142 |
| RA-YZ-F | aacttcgaagcagctccagcctaca | NO: 143 |
| RA-YZ-R | atgattacgccaagtttgcacgcct | NO: 144 |
| thrA-YZ-F | ACATAAGCGCGAATAATGAC | NO: 145 |
| thrA-YZ-R | cgccctgagtgcttgcggcagcgtg | NO: 146 |
| ApaI-LA-metL | CGCGCGGATCTTCCAGAGATTGGGCCCACGTAG | NO: 147 |
| | TGCAGCGCG | |
| metL-LA-EcoRI | AAGCTTGACGTCCAGGTGCCTCTTAAGGCTTAC | NO: 148 |
| | CTCGTTGCCGC | |
| KpnI-RA-metL | CACGCTAGCGGATCCGAGCTCGGTACCTCCTCT | NO: 149 |
| | GCGGGCGCG | |
| metL-RA-SalI | CGTGCGGACGGCAAGCTGCTACAGCTGTCCATG | NO: 150 |
| | GCGATGTTGGCG | |
| LA-YZ-F | tgaattagaactcggtacgcgcgga | NO: 151 |
| LA-YZ-R | tagagaataggaacttcgaactgca | NO: 152 |
| RA-YZ-F | aacttcgaagcagctccagcctaca | NO: 153 |
| RA-YZ-R | atgattacgccaagtttgcacgcct | NO: 154 |
| metL-YZ-F | GAACGACGACGAACAGTA | NO: 155 |
| metL-YZ-R | cgccctgagtgcttgcggcagcgtg | NO: 156 |
| ApaI-LA-proC | CGCGCGGATCTTCCAGAGATTGGGCCCAACGTT | NO: 157 |
| | GGCCCGCAG | |
| proC-LA-EcoRI | AAGCTTGACGTCCAGGTGCCTCTTAAGCAAATC | NO: 158 |
| | CCTCGTCTGTGTGT | |
| KpnI-RA-proC | CACGCTAGCGGATCCGAGCTCGGTACCATAACC | NO: 159 |
| | CCGAGTTAAGGAGAAG | |
| proC-RA-SalI | CGTGCGGACGGCAAGCTGCTACAGCTGTCCACG | NO: 160 |
| | CCCAGATCGG | |
| LA-YZ-F | tgaattagaactcggtacgcgcgga | NO: 161 |
| LA-YZ-R | tagagaataggaacttcgaactgca | NO: 162 |
| RA-YZ-F | aacttcgaagcagctccagcctaca | NO: 163 |
| RA-YZ-R | atgattacgccaagtttgcacgcct | NO: 164 |
| proC-YZ-F | CCAGCGTGGTGGACTTG | NO: 165 |
| proC-YZ-R | cgccctgagtgcttgcggcagcgtg | NO: 166 |
| ApaI-LA-dapA | CGCGCGGATCTTCCAGAGATTGGGCCCGAAGAT | NO: 167 |
| | CCTGCAGGATGCC | |
| dapA-LA-EcoRI | AAGCTTGACGTCCAGGTGCCTCTTAAGAAAATA | NO: 168 |
| | TTCCTTCTGTGT | |
| KpnI-RA-dapA | CACGCTAGCGGATCCGAGCTCGGTACCATTTGTT | NO: 169 |
| | AGCTAAGCCGC | |
| dapA-RA-SalI | CGTGCGGACGGCAAGCTGCTACAGCTGCGGAG | NO: 170 |
| | GAGGCCTGGC | |
| LA-YZ-F | tgaattagaactcggtacgcgcgga | NO: 171 |
| LA-YZ-R | tagagaataggaacttcgaactgca | NO: 172 |
| RA-YZ-F | aacttcgaagcagctccagcctaca | NO: 173 |
| RA-YZ-R | atgattacgccaagtttgcacgcct | NO: 174 |
| dapA-YZ-F | GGCGCATTCTCTGCGCG | NO: 175 |
| dapA-YZ-R | cgccctgagtgcttgcggcagcgtg | NO: 176 |

Shake flask culture was conducted to verify spermidine yield of the strains S5-1, S5-2, and S5-3. A highest spermidine production was measured in the supernatant of S5-3 shake flask, with a spermidine concentration of 23.3 g/L.

### EXAMPLE 6

### Knockout of Key Enzymes in Degradation Pathway in the Genetically Engineered Strain of Serratia marcescens Pathway

Utilization and degradation pathways (competitive pathways) for Arginine (Arg): (1) Arg was degraded into N²-succinylarginine through arginine N-succinyltransferase encoded by the astA gene (with an amino acid sequence available under an NCBI accession number TEW83071.1), and was further degraded into L-glutamate (L-Glu) and succinate through ast series of genes. To reduce the degradation of Arg, the Arg degradation pathway was weakened by deleting the astA gene.

Following the Red homologous recombination method described in Example 2, the astA gene was deleted based on the strain S5-3 as a parental strain for the metabolic pathway to prevent further degradation of Arg and redirect more metabolic flux toward spermidine synthesis. A strain S5-3ΔastA was constructed, which was designated as a strain S6. Primers used are shown in Table 8 below.

TABLE 8
Primer sequences (SEQ ID NO: 177-186)

| Primer name | Sequence 5′-3′ | SEQ |
|---|---|---|
| ApaI-LA-astA | CGCGCGGATCTTCCAGAGATTGGGCCC | NO: 177 |
| | GGCCAAAAAACTGATCGAC | |
| astA-LA-EcoRI | AAGCTTGACGTCCAGGTGCCTCTTAAG | NO: 178 |
| | AATGCGAACCTCTTCTGAATCA | |
| KpnI-RA-astA | CACGCTAGCGGATCCGAGCTCGGTACC | NO: 179 |
| | GTGCTCCCCCTTATTGCACA | |
| astA-RA-SalI | CGTGCGGACGGCAAGCTGCTACAGCTA | NO: 180 |
| | CCACCCCGCCCATGAA | |
| LA-YZ-F | tgaattagaactcggtacgcgcgga | NO: 181 |
| LA-YZ-R | tagagaataggaacttcgaactgca | NO: 182 |
| RA-YZ-F | aacttcgaagcagctccagcctaca | NO: 183 |
| RA-YZ-R | atgattacgccaagtttgcacgcct | NO: 184 |
| astA-YZ-F | GTCCCGGTTTATGCCCCT | NO: 185 |
| astA-YZ-R | cgccctgagtgcttgcggcagcgtg | NO: 186 |

Shake flask culture was conducted to verify spermidine yield of the strain S6. A concentration of spermidine in the supernatant in the shake flask was determined to be 26.8 g/L.

### EXAMPLE 7

### Introduction of an Exogenous Spermidine Synthetic Pathway—Genetically Engineered Strain of E. coli

Aspartate semialdehyde is an important intermediate metabolite in cells, and serves as a direct precursor for the synthesis of methionine and decarboxylated S-adenosylmethionine, is also an important precursor for spermidine synthesis. The metabolic pathway from aspartate semialdehyde to decarboxylated S-adenosylmethionine is complex and involves a long pathway and the metabolism of multiple amino acid intermediates. Excessive modification of the metabolic pathway can easily affect bacterial growth. Based on the above reasons, this example introduces the CAPADH/CAPADC/APAUH pathway derived from different microbial strains into E. coli for heterologous expression. A schematic diagram of the construction is shown in the figure.

The carboxyaminopropylagmatine dehydrogenase encoded by CAPADH directly uses aspartate semialdehyde and agmatine as precursors to synthesize carboxyaminopropylagmatine. It is then decarboxylated to generate aminopropylagmatine under the action of carboxyaminopropylagmatine decarboxylase encoded by CAPADC, and finally to generate spermidine under the action of aminopropylagmatine ureahydrolase encoded by APAUH. First, various carboxyaminopropylagmatine dehydrogenases (CAPADH), carboxyaminopropylagmatine decarboxylases (CAPADC), and aminopropylagmatine ureahydrolases (APAUH) from different microbial sources were expressed in E. coli via plasmids. An enzyme with highest catalytic efficiency was further selected, and the CAPADH/CAPADC/APAUH pathway was exogenously integrated through homologous recombination. A genomic integration site selected in the present disclosure was located upstream of an argA gene in the E. coli BL21.

The specific procedure process is as follows:

Using amplification primers, CAPADH genes stcapadh, dacapadh, gscapadh and iccapadh were cloned from genomes of Spirochaeta thermophila DSM 6578, Denitrovibrio acetiphilus DSM 12809, Geobacter sulfurreducens KN400 and Ilumatobacter coccineus YM16-304, respectively, and amino acid sequences of the cloned genes were listed in the NCBI database under accession numbers of WP_014623968.1, WP_013011851.1, WP_010943175.1, and WP_015442124.1, respectively; CAPADC genes stapauh, daapauh, lbapauh and icapauh were cloned from genomes of Spirochaeta thermophila DSM 6578, Denitrovibrio acetiphilus DSM 12809, Geobacter sulfurreducens KN400 and Ilumatobacter coccineus YM16-304, respectively, and amino acid sequences of the cloned genes were listed in the NCBI database under accession numbers of WP_014623969.1, WP_013011852.1, WP_004513933.1 and WP_015442123.1, respectively; and APAUH genes stapauh, daapauh, lbapauh and icapauh were cloned from genomes of Spirochaeta thermophila DSM 6578, Denitrovibrio acetiphilus DSM 12809, Geobacter sulfurreducens KN400 and Ilumatobacter coccineus YM16-304, respectively, and amino acid sequences of the cloned genes were listed in the NCBI database under accession numbers of WP_014623970.1, WP_013011853.1, WP_015770028.1 and WP_015442122.1, respectively. The three types of cloned genes were ligated into a same plasmid pRSFDuet-1 to obtain recombinant E. coli with enhanced expression of the three genes.

The CAPADH and CAPADC in the aforementioned microorganisms are located adjacent to each other within a gene cluster. Using stcapadh-BamHI-F and stcapadc-HindIII-R as primers, and Spirochaeta thermophila DSM 6578 genome as a template, a target fragment stcapadh-stcapadc was amplified; and a first multiple cloning site of the pRSFDuet-1 plasmid was digested with HindIII and BamHI. After purification of the digestion product, the digested plasmid was ligated with the amplified target fragment stcapadh-stcapadc, the ligated product pRSFDuet-1-stcapadh-stcapadc was transformed into competent JM109 cells. Positive clones were selected on kanamycin-resistant plates and further verified by colony PCR. Using stapauh-KpnI-F and stapauh-XhoI-R as primers, and Spirochaeta thermophila DSM 6578 genome as a template, a target fragment stapauh was amplified; and a second multiple cloning site of the pRSFDuet-1-stcapadh-stcapadc was digested with KpnI and XhoI. After purification of the digestion product, the digested plasmid was ligated with the amplified target fragment stapauh, the ligated product pRSFDuet-1-stcapadh-stcapadc-stapauh was transformed into competent JM109 cells. Positive clones were selected on kanamycin-resistant plates and further verified by colony PCR. The JM109 strain that was successfully ligated was selected, and the plasmid pRSFDuet-1-stcapadh-stcapadc-stapauh was extracted and transformed into competent cells E. coli BL21 to construct a spermidine synthesis strain Escherichia coli-stcapadh-stcapadc-stapauh. The construction methods for other strains were similar to that of the Escherichia coli-stcapadh-stcapadc-stapauh. Specifically, CAPADH, CAPADC, and APAUH from different strains were ligated into the two multiple cloning sites of the plasmid pRSFDuet-1. The ligation and methods of other genes were similar to the above. The primer sequences used are listed in Table 9.

TABLE 9
Primer sequences (SEQ ID NO: 188-191)

| Primer name | Sequence 5′-3′ | SEQ ID |
|---|---|---|
| stcapadh-BamHI-F | CCACAGCCAGGATCCGATGGGCACG | NO: 188 |
| | GGTGCTCATCAT | |
| stcapadc-HinIII-R | GCATTATGCGGCCGCAAGCTTTTAC | NO: 189 |
| | GAGAGGCGCCGCTT | |
| stapauh-KpnI-F | GCGATCGCTGACGTCGGTACCATGC | NO: 190 |
| | GCTATCCCCACTTCCT | |
| stapauh-XhoI-R | GGTTTCTTTACCAGACTCGAGCTAC | NO: 191 |
| | CGCCGGGGTAGATGCC | |

Recombinant E. coli was inoculated into a shake-flask fermentation medium at a volume ratio of 2%. When the cells reached an OD₆₀₀ of 0.6-0.8, IPTG was added to a final concentration of 0.4 mM to induce gene expression at 20° C. for 12 hours. Upon completion of induction, a supernatant was collected with centrifugation at 8000 rpm at 4° C. for 20 minutes. A spermidine concentration in the supernatant was determined by high-performance liquid chromatography (HPLC), as shown in Table 10.

TABLE 10

| Recombinant strains | Spermidine (g/L) |
|---|---|
| E. coli-stcapadh-stcapadc-stapauh | 6.7 |
| E. coli-iccapadh-iccapadc-icapauh | 4.3 |
| E. coli-dacapadh-dacapadc-daapauh | 2.1 |
| E. coli-gscapadh-gscapadc-lbapauh | 3.7 |
| E. coli-stcapadh-gscapadc-stapauh | 4.1 |
| E. coli-gscapadh-stcapadc-stapauh | 5.4 |
| E. coli-stcapadh-dacapadc-stapauh | 3.2 |
| E. coli-dacapadh-stcapadc-stapauh | 1.2 |

Overexpressing key genes involved in spermidine synthesis via expression vectors imposes a metabolic burden on the bacterial cells, thereby affecting their growth. The expression vectors suffered from the structural instability and easy loss during proliferation of the bacterial cells, as well as fluctuations in plasmid copy number, in addition, the use of antibiotics inhibited cell growth and posed environmental contamination risks, while IPTG induction increased production costs, thus limiting the scalability of production. Therefore, the recombinant strain Escherichia coli-stcapadh-stcapade-stapauh, which demonstrated the best effect of spermidine synthesis, was selected. An exogenous gene expression fragment was constructed by fusing a promoter J23119 in the E. coli and a ribosome binding site (RBS) B0034 with the gene stcapadh-stcapade-stapauh. According to the aforesaid section “4. Gene integration and expression scheme”, the exogenous gene expression fragment was integrated into a knockout strain E. coli BL21 to construct a strain Escherichia coli-stcapadh-stcapade-stapauh, designated as a strain E1.

Shake flask culture was conducted to verify spermidine yield of the strain E1. A concentration of spermidine in the supernatant in the shake flask was determined to be 6.7 g/L.

### EXAMPLE 8

### Relief of Feedback Inhibition of Key Enzymes in the Genetically Engineered Strain of E. coli Pathway and Knockout of Repressor Protein

In the synthesis of key intermediates in aspartate semialdehyde and arginine, a repressor protein inhibits the synthesis of both intermediates and final products. In the synthesis of key intermediates in aspartate semialdehyde and arginine, a repressor protein inhibited the synthesis of both intermediates and final products. An argR gene (with an amino acid sequence available under an NCBI accession number QJZ05382.1) encoded a DNA-binding dual transcriptional regulator that repressed the expression of an arg gene series, and inhibited the excessive synthesis of key intermediates such as ornithine and arginine. Therefore, in order to ensure stable and high-rate synthesis of the intermediates, the gene encoding the repressor protein was deleted to increase a metabolic flux.

In the metabolic pathway of arginine synthesis, N-acetylglutamate synthase encoded by the argA gene (with an amino acid sequence available under an NCBI accession number QJZ04982.1) was subjected to feedback inhibition by its product arginine. In order to improve the enzyme activity, base mutation of the gene encoding the enzyme was further conducted on the basis of knocking out the argR gene to construct a negative feedback mutant, thereby releasing feedback inhibition and obtaining a resulting engineered strain E. coli argA^{fbr}ΔArgR, designated as a strain E2.

The specific procedure process is as follows:

### (1) Knockout of the Repressor Protein ArgR

Primer design is listed in Table 11 below:

TABLE 11
Primer sequences (SEQ ID NO: 192-201)

| Primer name | Primer sequence 5′-3′ | SEQ ID |
|---|---|---|
| ApaI-LA-argR | CGCGCGGATCTTCCAGAGATTGGGC | NO: 192 |
| | CCACCTGTGACAGCAGCGGCAG | |
| argR-LA-EcoRI | AAGCTTGACGTCCAGGTGCCTCTTA | NO: 193 |
| | AGAAGTCACCCGATATGGTGGT | |
| KpnI-RA-argR | CACGCTAGCGGATCCGAGCTCGGTA | NO: 194 |
| | CCTCTCTGCCCCGTCGTTTCTG | |
| argR-RA-SalI | CGTGCGGACGGCAAGCTGCTACAGC | NO: 195 |
| | TGCGATGCACGGCAGAACTCGC | |
| argR-LA-YZ-F | tgaattagaactcggtacgcgcgga | NO: 196 |
| argR-LA-YZ-R | tagagaataggaacttcgaactgca | NO: 197 |
| argR-RA-YZ-F | aacttcgaagcagctccagcctaca | NO: 198 |
| argR-RA-YZ-R | atgattacgccaagtttgcacgcct | NO: 199 |
| argR-YZ-F | AGACCCGCCACCGGCCTTCGCTTCA | NO: 200 |
| argR-YZ-R | Cgccctgagtgcttgcggcagcgtg | NO: 201 |

The plasmid PKD46, which expressed homologous recombinase, was extracted using a plasmid extraction kit, purified, and then transformed into competent cells of E1 to construct E1-PKD46. After being prepared by the CaCl₂ method, the competent cells of E1-PKD46 were stored at −80° C. for subsequent use. A plasmid T19, serving as a template plasmid for homologous recombination fragment construction, was extracted using a plasmid extraction kit and purified with a gel extraction kit. Primer pairs ApaI-LA-argR and argR-LA-EcoRI, as well as KpnI-RA-argR and argR-RA-SalI were designed to amplify the corresponding homologous arm left arm (LA) and right arm (RA) fragments, which were purified and recovered using a gel extraction kit. The plasmid T19 was digested with ApaI and EcoRI, and the digestion results were detected by agarose gel electrophoresis. A size of the digested fragment was 5041bp, and the digestion product was purified and recovered. The product after double-enzyme digestion of the T19 vector was ligated with the left arm (LA) fragment. The ligated product was transformed into competent E. coli JM109 cells, and plated on a kanamycin-resistant plate. Positive clones are screened and verified by colony PCR using primers LA-YZ-F and LA-YZ-R, producing a band with a size of approximately 1100 bp. Verified clones were sent for sequencing to further confirm successful ligation of the fragments. Subsequently, the plasmid containing the LA fragment was digested with KpnI and SalI. The RA fragment was ligated using the same method, and the ligated plasmid was verified. The plasmid containing both the LA and RA fragments was extracted and used as a template for PCR with primers ApaI-LA-argR and argR-RA-SalI to obtain a target fragment argR-LA-FRT-Kan-FRT-RA. Agarose gel electrophoresis confirmed an expected size of the PCR product was approximately 3324 bp. After the PCR product was purified and treated with the restriction enzyme DpnI to eliminate template effect. After digestion with the restriction enzyme DpnI, the PCR product of the target fragment argR-LA-FRT-Kan-FRT-RA PCR was further purified and then transformed into competent cells E. coli BL21-PKD46. Positive transformants were screened on resistance plates using a kanamycin resistance marker. In order to recombination in the screened positive transformants had occurred, primers argR-YZ-F and argR-YZ-R were used as templates. When a fragment of approximately 1100 bp was amplified, it was confirmed that recombination had occurred. Otherwise, when no fragment was amplified, a false-positive clone was confirmed. When verifying whether recombination had occurred by PCR amplification, the corresponding PCR verification bacteria were spotted on ampicillin and kanamycin dual-resistance plates. A correct transformant E1-PKD46 (kana^{R}) that was confirmed to have undergone recombination was subjected to elimination by a kanamycin resistance marker. First, competent cells of E1-PKD46 (kana^{R}) were prepared, and the extracted plasmid pCP20 was transformed into the competent cells of E1-PKD46 (kana^{R}). Transformants in which the resistance had been eliminated were screened using both non-resistant and resistant plates. Colony PCR was then performed, and a transformant E1-ΔargR with knockout of the argR gene and eliminated resistance were screened.

(2) Construction of argA^{fbr} Mutant

The argA gene was subjected to feedback inhibition by arginine, the target product. In the present disclosure, a point mutation was introduced at a 43rd nucleotide of the argA gene, changing a cytosine (C) to thymine (T), and the encoded histidine was converted into tyrosine, thereby significantly increasing arginine production. Therefore, the mutant gene at the site was integrated into a plasmid pRSFDuet-1.

The specific procedure process is as follows:

Primer design is listed in Table 12 below:

TABLE 12
Primer sequences (SEQ ID NO: 202-205)

| Primer name | Primer sequence 5′-3′ | SEQ ID |
|---|---|---|
| F-argA- | GTGGTAAAGGAACGTAAAACCGA | NO: 202 |
| amplification | | |
| primer | | |
| R-argA- | TTACCCTAAATCCGCCATCAACAC | NO: 203 |
| amplification | | |
| primer | | |
| argA-TB-F | GTCGAGGGATTCCGCTATTCGGTTCC | NO: 204 |
| argA-TB-R | AGCGGAATCCCTCGACCAACTCGGT | NO: 205 |

First, a plasmid pRSFDuet-1 (high-copy) was extracted using a plasmid extraction kit, and was subjected to double digestion with BamHI and HindIII. A restriction digestion system (50 μL) was as follows: 5 μL 10×Q Cut Buffer, plasmid 20 μL, 1 μL BamHI, 1 μL HindIII, and 33 μL ddH₂O. After digestion, the product was purified. Next, a wild-type E. coli BL21 genome was extracted and used as a template to amplify the argA gene fragment. After verification, the PCR product was ligated into the digested plasmid pRSFDuet-1. Competent E. coli JM109 cells were prepared using a CaCl₂ method. The plasmid pRSFDuet-1 carrying the argA fragment was then transformed into the competent cells. Positive transformants were selected and identified by colony PCR as the successfully transformed strain E. coli JM109-argA. A plasmid pRSFDuet1-argA was extracted from E. coli JM109-argA and used as a template for site-directed mutagenesis of argA. Mutant genes were designed and extracted using a Fast Mutangenesis System kit. The extracted plasmid pRSFDuet1-metA was used as a template, and an argA^{fbr} fragment was amplified using the argA-TB-F and argA-TB-R. A PCR amplification system (50 μL) was as follows: 3 μL plasmid template, 1 μL argA-TB-F, 1 μL argA-TB-R, 25 μL 2×TransStart®FastPfu Fly PCR SuperMix, and 20 μL ddH₂O. PCR amplification program was as follows: 94° C. for 2-5 minutes, 20-25 cycles (94° C. for 20 seconds, 55° C. for 20 seconds, 72° C. at 2-6 kb/min), 72° C. for 10 minutes, and storage at 4° C. The resulting PCR product was digested with 1-3 μL of DMT enzyme, mixed well, and incubated at 37° C. for 1 hour. The DMT digestion product was then purified using a PCR product purification kit. 2-5 μL was taken and added to 50 μL of competent JM109 cells for transformation. Positive transformants were selected and identified by colony PCR, and then sequenced by a sequencing company to identify the correct transformant, JM109-argA^{fbr}.

The plasmid pRSFDuet1-argA^{fbr} was extracted from the transformant JM109-argA^{fbr}. According to the aforesaid section “4. Gene integration and expression scheme”, the argA^{fbr} fragment was integrated into the knockout strain E1-ΔargR to construct the recombinant strain E1-argA^{fbr}ΔArgR, designated as strain E2, for subsequent studies.

Shake flask culture was conducted to verify arginine production of the strain E2. A concentration of arginine in the supernatant in the shake flask was determined to be 7.8 g/L.

### EXAMPLE 9

### Enhanced Expression of Key Enzymes in the Pathway

In the metabolic processes of synthesizing important intermediates such as decarboxylated aspartate semialdehyde and arginine, some genes, acting as rate-limiting enzymes, strictly regulate the metabolic flux of the metabolic pathway. The N-acetylglutamate synthase encoded by the argA gene functions as a rate-limiting enzyme in the arginine metabolic synthesis pathway. The arginine succinate lyase encoded by the argH gene catalyzes the synthesis of arginine from arginine succinate. The two enzymes significantly inhibit the excessive synthesis of arginine. The arginine decarboxylase encoded by the speA gene catalyzes the conversion of arginine to agmatine, which is a critical precursor in the novel pathway for the production of spermidine. Therefore, expression of key enzymes involved in the arginine was enhanced by replacing a native promoter with a strong trc promoter, thereby increasing metabolic flux toward arginine synthesis. The trc promoter SEQ ID NO:187: 5′-TTGACAATTAATCATCCGGCTCGTATAATG-3′) was derived from a plasmid pTrc99a. The aspartate kinase encoded by the lysC gene catalyzed the phosphorylation of aspartate. The aspartate semialdehyde dehydrogenase encoded by asd gene catalyzed the phosphorylated aspartate to generate aspartate semialdehyde. In order to enhance the enzyme activity and flux of the metabolic pathway, the native promoter of the gene was replaced with the strong trc promoter to promote higher gene expression and increased production of the desired product.

Using the strain E2 as a parental strain, the expression of the following key enzymes in the synthetic pathway was enhanced: lysC (with an amino acid sequence available under an NCBI accession number QZI64953.1), asd (with an amino acid sequence available under an NCBI accession number QJZ05564.1), argA (with an amino acid sequence available under an NCBI accession number QJZ04982.1), argH (with an amino acid sequence available under an NCBI accession number QJZ06087.1) and speA (with an amino acid sequence available under an NCBI accession number QJZ05082.1). By replacing the native promoter of the gene with the strong trc promoter, the engineered strain, designated as a strain E2-P_{trc}lysc-P_{trc}asd-P_{trc}argA-P_{trc}argH-P_{trc}speA, was obtained.

The specific procedure process is as follows:

Based on the lysC of E. coli BL21 in NCBI, primers were designed using Primer Premier 5 (see Table 13). A target gene fragment, lysC, was amplified by PCR using E. coli BL21 genome as a template and lysC-KZ-F/R as primers, with EcoRI and HindIII restriction sites added upstream and downstream, respectively, The PCR fragment was purified. The plasmid pTrc99a was digested with EcoRI and HindIII, purified, and ligated with the purified lysC fragment for transformation into competent E. coli JM109 cells. Positive transformants were screened and verified by colony PCR using verification primers lysC-99a-YZ-F/R, and further confirmed by sequencing. The successfully transformed plasmid pTrc-lysC was extracted and used as a template. The target fragment trc-lysC was amplified using BamHI-trc-lysC-F and trc-lysC-SacI-R as primers. The target fragment was verified to be approximately 1453 bp by agarose gel electrophoresis and purified. The T19 vector plasmid was digested with BamHI and SacI, purified, and ligated with the trc-lysC fragment for transformation. The ligated product was verified by colony PCR with primers trc-lysC-T19-YZF/YZR, followed by sequencing, and the correct positive transformant T19-trc-lysC was selected. Using the lysC genome in E. coli BL.21 as a template, the left arm (LA) fragment was amplified using lysC-LA-ApaI and EcoRI-lysC-LA as primers, and the right arm (RA) fragment was amplified using lysC-RA-KpnI and SalI-lysC-RA as primers. After purification, the plasmid was ligated to the left arm and the right arm of the double-digested and purified plasmid T19-trc-lysc, respectively. The ligated product was verified by colony PCR with verification primers lysC-LA-YZ-F and lysC-LA-YA-R, as well as lysC-RA-YZ-F and lysC-RA-YA-R followed by sequencing to identify to the plasmid T19-LA-trc-lysC-RA with the left arm and the right arm correctly ligated. The recombinant plasmid T19-LA-trc-lysC-RA, with the left arm and the right arm correctly ligated, was extracted and used as the template to amplify the target fragment LA-trc-lysC-RA using lysC-LA-ApaI and SalI-lysC-RA as primers. The fragment was approximately 3700 bp in size. The PCR product was purified and treated with the restriction enzyme DpnI to remove template effect. The digested product was further purified and then transformed into E2-PKD46 (the homologous recombinase expression plasmid PKD46 is extracted using a plasmid extraction kit, purified, and transformed into competent E2 cells to construct E2-PKD46) competent cells. Positive transformants were screened on resistance plates using a kanamycin resistance marker. When recombination had occurred, a fragment of approximately 1000 bp could be amplified using the primers lysC-YZ-F and lysC-YZ-R; otherwise, no fragment could be amplified. In addition, the positive transformants were verified on ampicillin and kanamycin dual-resistance plates using the corresponding PCR method, confirming that the correct transformants E2-PKD46 (kana^{R}) was obtained. Kanamycin resistance was eliminated. First, competent cells of E2-PKD46 (kana^{R}) were prepared using the CaCl₂ method, and the plasmid pCP20 was transformed into the competent cells of E2-PKD46 (kana^{R}). Transformants in which the resistance had been eliminated were screened using both non-resistant and resistant plates. Colony PCR was then performed to identify the transformant E2-P_{trc}lysC that replaced the lysC gene promoter and eliminated resistance.

Following the same strategy, the promoters of the genes asd, argA, argH and speA were replaced in succession. A strain E2-P_{trc}lysC-P_{trc}asd-P_{trc}argA-P_{trc}argH-P_{trc}speA was constructed, which was designated as E3. Primers used are shown in Table 13 below.

TABLE 13
Primer sequences (SEQ ID NO: 206-295)

| Primer name | Primer sequence 5′-3′ | SEQ ID |
|---|---|---|
| LysC-KZ-F | TGGAATTCATGTCTGAAATTGTTGTCTCCA | NO: 206 |
| LysC-KZ-R | GCCAAGCTTTTACTCAAACAAATTACTATGCAG | NO: 207 |
| lysC-99a-YZ-F | TCACTGCATAATTCGTGTCGCTCAA | NO: 208 |
| lysC-99a-YZ-R | CGCTACTGCCGCCAGGCAAATTCTG | NO: 209 |
| BamHI-trc-lysC-F | AGCCTACACGCTAGCGGATCCTTGACAATTAATCA | NO: 210 |
| | TCCGGC | |
| trc-lysC-SacI-R | AGTATCTCAGGTACCGAGCTCTTACTCAAACAAAT | NO: 211 |
| | TACTATGCAG | |
| trc-lysC-T19-YZF | tcttctaataaggggatcttgaagt | NO: 212 |
| trc-lysC-T19-YZR | cgctgttgcggatcatctccagcgt | NO: 213 |
| lysC-LA-Apal | CGCGCGGATCTTCCAGAGATTGGGCCCTCAGAAC | NO: 214 |
| | CGCCGATCCCGA | |
| EcoRI-lysC-LA | AAGCTTGACGTCCAGGTGCCTCTTAAGAACTACC | NO: 215 |
| | TCGTGTCAGGGGA | |
| lysC-RA-KpnI | CACGCTAGCGGATCCGAGCTCGGTACCATACTGT | NO: 216 |
| | ATGGCCTGGAAGC | |
| SalI-lysC-RA | CGTGCGGACGGCAAGCTGCTACAGCTGCCGCATT | NO: 217 |
| | ACCCTGGTGCAG | |
| lysC-LA-YZ-F | tgaattagaactcggtacgcgcgga | NO: 218 |
| lysC-LA-YZ-R | tagagaataggaacttcgaactgca | NO: 219 |
| lysC-RA-YZ-F | aacttcgaagcagctccagcctaca | NO: 220 |
| lysC-RA-YZ-R | atgattacgccaagtttgcacgcct | NO: 221 |
| LysC-YZ-F | TCAGAACCGCCGATCCCGAT | NO: 222 |
| LysC-YZ-R | Cgccctgagtgcttgcggcagcgtg | NO: 223 |
| asd-KZ-F | AGGAAACAGACCATGGAATTCATGAAAAATGTTG | NO: 224 |
| | GTTTTAT | |
| asd-KZ-R | TCCGCCAAAACAGCCAAGCTTTTACGCCAGTTGA | NO: 225 |
| | CGAAGCA | |
| asd-99a-YZ-F | TCACTGCATAATTCGTGTCGCTCAA | NO: 226 |
| asd-99a-YZ-R | CGCTACTGCCGCCAGGCAAATTCTG | NO: 227 |
| BamHI-trc-asd-F | AGCCTACACGCTAGCGGATCCTTGACAATTAATCA | NO: 228 |
| | TCCGGC | |
| trc-asd-SacI-R | ATCAGTATCTCAGGTACCGAGCTCTTACGCCAGTT | NO: 229 |
| | GACGAAGCA | |
| trc-asd-T19-YZF | tcttctaataaggggatcttgaagt | NO: 230 |
| trc-asd-T19-YZR | cgctgttgcggatcatctccagcgt | NO: 231 |
| asd-LA-ApaI | CGCGCGGATCTTCCAGAGATTGGGCCCTTGTTGT | NO: 232 |
| | CAGCCACGTTAAC | |
| EcoRI-asd-LA | AAGCTTGACGTCCAGGTGCCTCTTAAGAAGCGTT | NO: 233 |
| | TTTTTCCTGCAAA | |
| asd-RA-KpnI | CACGCTAGCGGATCCGAGCTCGGTACCTCTTTATT | NO: 234 |
| | CATTAAATCTGGG | |
| SalI-asd-RA | CGTGCGGACGGCAAGCTGCTACAGCTGATTGGCG | NO: 235 |
| | CATCAAACTGATT | |
| asd-LA-YZ-F | tgaattagaactcggtacgcgcgga | NO: 236 |
| asd-LA-YZ-R | tagagaataggaacttcgaactgca | NO: 237 |
| asd-RA-YZ-F | aacttcgaagcagctccagcctaca | NO: 238 |
| asd-RA-YZ-R | atgattacgccaagtttgcacgcct | NO: 239 |
| asd-YZ-F | TTGTTGTCAGCCACGTTAAC | NO: 240 |
| asd-YZ-R | Cgccctgagtgcttgcggcagcgtg | NO: 241 |
| argA-KZ-F | AGGAAACAGACCATGGAATTCGTGGTAAAGGAA | NO: 242 |
| | CGTAAAAC | |
| argA-KZ-R | TCCGCCAAAACAGCCAAGCTTTTACCCTAAATCC | NO: 243 |
| | GCCATCA | |
| argA-99a-YZ-F | TCACTGCATAATTCGTGTCGCTCAA | NO: 244 |
| argA-99a-YZ-R | CGCTACTGCCGCCAGGCAAATTCTG | NO: 245 |
| BamHI-trc-argA-F | AGCCTACACGCTAGCGGATCCTTGACAATTAATCA | NO: 246 |
| | TCCGGC | |
| trc-argA-SacI-R | ATCAGTATCTCAGGTACCGAGCTCTTACCCTAAAT | NO: 247 |
| | CCGCCATCA | |
| trc-argA-T19-YZF | tcttctaataaggggatcttgaagt | NO: 248 |
| trc-argA-T19-YZR | cgctgttgcggatcatctccagcgt | NO: 249 |
| argA-LA-ApaI | CGCGCGGATCTTCCAGAGATTGGGCCCTACGCGC | NO: 250 |
| | ACTTGCAATGGAA | |
| EcoRI-argA-LA | AAGCTTGACGTCCAGGTGCCTCTTAAGGGCACAC | NO: 251 |
| | CTCTTTGCATGAT | |
| argA-RA-KpnI | CACGCTAGCGGATCCGAGCTCGGTACCGCGATGA | NO: 252 |
| | AAATCGTCGGATG | |
| SalI-argA-RA | CGTGCGGACGGCAAGCTGCTACAGCTGGATCGTG | NO: 253 |
| | ATCAACTGGCCTC | |
| argA-LA-YZ-F | tgaattagaactcggtacgcgcgga | NO: 254 |
| argA-LA-YZ-R | tagagaataggaacttcgaactgca | NO: 255 |
| argA-RA-YZ-F | aacttcgaagcagctccagcctaca | NO: 256 |
| argA-RA-YZ-R | atgattacgccaagtttgcacgcct | NO: 257 |
| argA-YZ-F | GCTGTTTCTGGGCTTTTGCT | NO: 258 |
| argA-YZ-R | Cgccctgagtgcttgcggcagcgtg | NO: 259 |
| ArgH-KZ-F | AGGAAACAGACCATGGAATTCATGGCACTTTGGG | NO: 260 |
| | GCGGGCG | |
| ArgH-KZ-R | TCCGCCAAAACAGCCAAGCTTTTACCCTAACCGA | NO: 261 |
| | GCCTGCG | |
| argH-99a-YZ-F | TCACTGCATAATTCGTGTCGCTCAA | NO: 262 |
| argH-99a-YZ-R | CGCTACTGCCGCCAGGCAAATTCTG | NO: 263 |
| BamHI-trc-argH-F | AGCCTACACGCTAGCGGATCCTTGACAATTAATCA | NO: 264 |
| | TCCGGC | |
| trc-argH-SacI-R | ATCAGTATCTCAGGTACCGAGCTCTTACCCTAACC | NO: 265 |
| | GAGCCTGCG | |
| trc-argH-T19-YZF | tcttctaataaggggatcttgaagt | NO: 266 |
| trc-argH-T19-YZR | cgctgttgcggatcatctccagcgt | NO: 267 |
| argH-LA-ApaI | CGCGCGGATCTTCCAGAGATTGGGCCCGTTGGGC | NO: 268 |
| | TGCCATTTTGCGA | |
| EcoRI-argH-LA | AAGCTTGACGTCCAGGTGCCTCTTAAGAACTCTG | NO: 269 |
| | TTTCCTTATTTTG | |
| argH-RA-KpnI | CACGCTAGCGGATCCGAGCTCGGTACCGAACATT | NO: 270 |
| | TATATGTATAAAT | |
| SalI-argH-RA | CGTGCGGACGGCAAGCTGCTACAGCTGACAGCC | NO: 271 |
| | AGCGCTGGCAGTAA | |
| argH-LA-YZ-F | tgaattagaactcggtacgcgcgga | NO: 272 |
| argH-LA-YZ-R | tagagaataggaacttcgaactgca | NO: 273 |
| argH-RA-YZ-F | aacttcgaagcagctccagcctaca | NO: 274 |
| argH-RA-YZ-R | atgattacgccaagtttgcacgcct | NO: 275 |
| ArgH-YZ-F | TCGGGTGTGACCCAGGCGCAAGT | NO: 276 |
| ArgH-YZ-R | Cgccctgagtgcttgcggcagcgtg | NO: 277 |
| speA-KZ-F | AGGAAACAGACCATGGAATTCATGTCTGACGACA | NO: 278 |
| | TGTCTATG | |
| speA-KZ-R | TCCGCCAAAACAGCCAAGCTTTTACTCATCTTCA | NO: 279 |
| | AGATAAG | |
| speA-99a-YZ-F | TCACTGCATAATTCGTGTCGCTCAA | NO: 280 |
| speA-99a-YZ-R | CGCTACTGCCGCCAGGCAAATTCTG | NO: 281 |
| BamHI-trc-speA-F | AGCCTACACGCTAGCGGATCCTTGACAATTAATCA | NO: 282 |
| | TCCGGC | |
| trc-speA-SacI-R | ATCAGTATCTCAGGTACCGAGCTCTTACTCATCTT | NO: 283 |
| | CAAGATAAG | |
| trc-speA-T19-YZF | tcttctaataaggggatcttgaagt | NO: 284 |
| trc-speA-T19-YZR | cgctgttgcggatcatctccagcgt | NO: 285 |
| speA-LA-ApaI | CGCGCGGATCTTCCAGAGATTGGGCCCGTGATCT | NO: 286 |
| | CTTCGATGTCTACC | |
| EcoRI-speA-LA | AAGCTTGACGTCCAGGTGCCTCTTAAGAGCGAAC | NO: 287 |
| | CTCAAATTATTTT | |
| speA-RA-KpnI | CACGCTAGCGGATCCGAGCTCGGTACCGTCCTGT | NO: 288 |
| | GTTACTTGAATCC | |
| SalI-speA-RA | CGTGCGGACGGCAAGCTGCTACAGCTGGCCAGA | NO: 289 |
| | GCAGTGATTTCCGA | |
| speA-LA-YZ-F | tgaattagaactcggtacgcgcgga | NO: 290 |
| speA-LA-YZ-R | tagagaataggaacttcgaactgca | NO: 291 |
| speA-RA-YZ-F | aacttcgaagcagctccagcctaca | NO: 292 |
| speA-RA-YZ-R | atgattacgccaagtttgcacgcct | NO: 293 |
| speA-YZ-F | GATGTCAGGAGACTGTTTGCCG | NO: 294 |
| sneA-YZ-R | Cgccctgagtgcttgcggcagcgtg | NO: 295 |

Shake flask culture was conducted to verify spermidine yield of the strain E3. A concentration of spermidine in the supernatant in the shake flask was determined to be 14.8 g/L.

### EXAMPLE 10

### Improving Transport Pathways

PotABCD is a transporter protein complex in E. coli responsible for the uptake of spermidine into the cell; therefore, it was knocked out. The PotABCD protein complex is composed of four subunits encoded by gene clusters potA, potB, potC, and potD. Specifically, the potA encodes an ATP-binding subunit, which provides the necessary energy for transport; the potB encodes an ABC transporter membrane subunit PotB; the potC encodes an ABC transporter membrane subunit PotC; and the potD encodes an ABC transporter periplasmic binding protein. Proper expression of all four genes in an operon is essential for functions of the operon. The loss of any single gene will inevitably result in functional inactivation. Therefore, in the present disclosure, the ATP-binding subunit gene potA (with an amino acid sequence available under an NCBI accession number QJZ03606.1) was deleted to weaken the capability of transporter proteins to transport spermidine. A strain with an improved transport pathway was constructed based on the strain E3. The PotA was knocked out using the Red homologous recombination method described in Example 2, and the resulting strain was designated as E3-ΔPotA. MdtJI (with an amino acid sequence available under an NCBI accession number CAQ32076.1) is a protein in E. coli responsible for exporting spermidine from the cells. It is encoded by a gene cluster consisting of mdtJ and mdtI. Therefore, its expression was enhanced using a strong trc promoter. Following the method described in Example 2, the promoter was replaced to obtain a strain E3-ΔPotA-P_{trc}MdtJI with enhanced transport pathway, which was designated as a strain E4. Primer sequences are shown in Table 14.

TABLE 14
Primer sequences (SEQ ID NO: 296-323)

| Primer name | Sequence 5′-3′ | SEQ |
|---|---|---|
| ApaI-LA-potA | CGCGCGGATCTTCCAGAGATTGGGCCCGGCT | NO: 296 |
| | TTCATCCGCCGGAACT | |
| potA-LA-EcoRI | AAGCTTGACGTCCAGGTGCCTCTTAAGGTAA | NO: 297 |
| | ACGCAACGGATGGCTT | |
| KpnI-RA-potA | CACGCTAGCGGATCCGAGCTCGGTACCGTTC | NO: 298 |
| | CAGAATGTAGTGATTG | |
| potA-RA-SalI | CGTGCGGACGGCAAGCTGCTACAGCTGTGT | NO: 299 |
| | CGTTGTTCATCAGCAGG | |
| LA-YZ-F | tgaattagaactcggtacgcgcgga | NO: 300 |
| LA-YZ-R | tagagaataggaacttcgaactgca | NO: 301 |
| RA-YZ-F | aacttcgaagcagctccagcctaca | NO: 302 |
| RA-YZ-R | atgattacgccaagtttgcacgcct | NO: 303 |
| potA-YZ-F | CGGTCGAAATCACGGATGATGTAGT | NO: 304 |
| potA-YZ-R | Cgccctgagtgcttgcggcagcgtg | NO: 305 |
| EcoRI-mdtJI-KZ-F | AGGAAACAGACCATGGAATTCATGTATATTT | NO: 306 |
| | ATTGGATTTT | |
| HindIII-mdtJI-KZ-R | TCCGCCAAAACAGCCAAGCTTTCAGGCAAG | NO: 307 |
| | TTTCACCATGA | |
| mdtJI-99a-YZ-F | TCACTGCATAATTCGTGTCGCTCAA | NO: 308 |
| mdtJI-99a-YZ-R | CGCTACTGCCGCCAGGCAAATTCTG | NO: 309 |
| BamHI-trc-metK-F | AGCCTACACGCTAGCGGATCCTTGACAATTA | NO: 310 |
| | ATCATCCGGC | |
| trc-mdtJI-SacI-R | AGTATCTCAGGTACCGAGCTCTCAGGCAAGT | NO: 311 |
| | TTCACCATGA | |
| trc-mdtJI-T19-YZF | tcttctaataaggggatcttgaagt | NO: 312 |
| trc-mdtJI-T19-YZR | cgctgttgoggatcatctccagcgt | NO: 313 |
| mdtJI-LA-ApaI | CGCGCGGATCTTCCAGAGATTGGGCCCCCCG | NO: 314 |
| | GGGATAATGAGAAACACTGTCA | |
| EcoRI-mdtJI-LA | AAGCTTGACGTCCAGGTGCCTCTTAAGTGTC | NO: 315 |
| | CTTCTCCTGCAAGAGA | |
| mdtJI-RA-KpnI | CACGCTAGCGGATCCGAGCTCGGTACCTGA | NO: 316 |
| | AGACGCTGCCCGCGCTG | |
| SalI-mdtJI-RA | CGTGCGGACGGCAAGCTGCTACAGCTGGGG | NO: 317 |
| | CCCTTTTATGCGCACTGATTACC | |
| mdtJI-LA-YZ-F | tgaattagaactcggtacgcgcgga | NO: 318 |
| mdtJI-LA-YZ-R | tagagaataggaacttcgaactgca | NO: 319 |
| mdtJI-RA-YZ-F | aacttcgaagcagctccagcctaca | NO: 320 |
| mdtJI-RA-YZ-R | atgattacgccaagtttgcacgcct | NO: 321 |
| MdtJI-YZ-F | GCCAGCAATCCCCAGACAAAAGCGA | NO: 322 |
| MdtJI-YZ-R | Cgccctgagtgcttgcggcagcgtg | NO: 321 |

Shake flask culture was conducted to verify spermidine yield of the strain E4. A concentration of spermidine in the supernatant in the shake flask was determined to be 18.5 g/L.

### EXAMPLE 11

### Weakening Competitive Pathways

(1) Homoserine Competitive Pathway:

An intermediate substance aspartate semialdehyde was further converted into homoserine by homoserine synthases encoded by thrA (with an amino acid sequence available under an NCBI accession number QJZ02606.1) and metL (with an amino acid sequence available under an NCBI accession number QJZ06068.1) genes. In order to eliminate the diversion of the competitive metabolic pathway, the thrA and metL genes were deleted based on the strain E4 using the Red homologous recombination method described in Example 1. A resulting strain E4-ΔthrAΔmetL was constructed, which was designated as a strain E5-1.

(2) Lys Competitive Pathways:

The dihydrodipicolinate synthase encoded by a dapA(with an amino acid sequence available under an NCBI accession number QJZ04698.1) gene converted the intermediate aspartate semialdehyde into dihydrodipicolinate. To mitigate the impact of this branching pathway and redirect more aspartate toward the metabolic pathway of aspartate semialdehyde, a strain E4-ΔthrAΔmetLΔdapA was constructed by knocking out the dapA gene based on the E4-ΔthrAΔmetL using the Red homologous recombination method described in Example 1. The resulting strain was designated as a strain E5-2.

(3) Glutamate-derived competitive pathways: Glutamate kinase encoded by proB and glutamate semialdehyde dehydrogenase encoded by proA, glutamate were converted to form glutamate semialdehyde. The glutamate semialdehyde was spontaneously converted to form pyrroline-5-carboxylate, which was then then reduced to proline (pro) by pyrroline-5-carboxylate reductase encoded by proC (with an amino acid sequence available under an NCBI accession number QJZ02915.1). Therefore, to block the metabolic flux in the competitive pathways, the proC gene was deleted to weaken the pathway. A strain E4-ΔthrAΔmetLΔdapAΔproC was constructed based on the strain E4-ΔthrAΔmetLΔdapA using the Red homologous recombination method described in Example 1 to delete the competitive pathway gene proC. The resulting strain was designated as a strain E5-3.

Primers used are shown in Table 15 below.

TABLE 15
Primer sequences (SEQ ID NO: 324-353)

| Primer name | Sequence 5′-3′ | SEQ |
|---|---|---|
| ApaI-LA-thrA | CGCGCGGATCTTCCAGAGATTGGGCCCCCAGAGCAG | NO: 324 |
| | AAAATATTGGG | |
| thrA-LA-EcoRI | AAGCTTGACGTCCAGGTGCCTCTTAAGGGTTGTTACC | NO: 325 |
| | TCGTTACCTTT | |
| KpnI-RA-thrA | CACGCTAGCGGATCCGAGCTCGGTACCCATGGTTAAA | NO: 326 |
| | GTTTATGCCC | |
| thrA-RA-SalI | CGTGCGGACGGCAAGCTGCTACAGCTGACCCCTGGG | NO: 327 |
| | TTACGGCTT | |
| LA-YZ-F | tgaattagaactcggtacgcgcgga | NO: 328 |
| LA-YZ-R | tagagaataggaacttcgaactgca | NO: 329 |
| RA-YZ-F | aacttcgaagcagctccagcctaca | NO: 330 |
| RA-YZ-R | atgattacgccaagtttgcacgcct | NO: 331 |
| thrA-YZ-F | AAGGGCTTTTTCTGCGACTT | NO: 332 |
| thrA-YZ-R | cgccctgagtgcttgcggcagcgtg | NO: 333 |
| ApaI-LA-metL | CGCGCGGATCTTCCAGAGATTGGGCCCCAACCCAACG | NO: 334 |
| | CGCGATGT | |
| metL-LA-EcoRI | AAGCTTGACGTCCAGGTGCCTCTTAAGTTTTACCCCT | NO: 335 |
| | TGTTTGCAGC | |
| KpnI-RA-metL | CACGCTAGCGGATCCGAGCTCGGTACCTTTCAGAAAT | NO: 336 |
| | TTAATAATGCC | |
| metL-RA-SalI | CGTGCGGACGGCAAGCTGCTACAGCTGTTCGCCTGTT | NO: 337 |
| | TAAAGTTAGA | |
| LA-YZ-F | tgaattagaactcggtacgcgcgga | NO: 338 |
| LA-YZ-R | tagagaataggaacttcgaactgca | NO: 339 |
| RA-YZ-F | aacttcgaagcagctccagcctaca | NO: 340 |
| RA-YZ-R | atgattacgccaagtttgcacgcct | NO: 341 |
| metL-YZ-F | TGCGTTGTCCCACCGATCCA | NO: 342 |
| metL-YZ-R | cgccctgagtgcttgcggcagcgtg | NO: 343 |
| ApaI-LA-proC | CGCGCGGATCTTCCAGAGATTGGGCCCGCCGCCGCCT | NO: 344 |
| | GTAGCGATAA | |
| proC-LA-EcoRI | AAGCTTGACGTCCAGGTGCCTCTTAAGTGCCTCACTC | NO: 345 |
| | CTGCCGTGAA | |
| KpnI-RA-proC | CACGCTAGCGGATCCGAGCTCGGTACCTGACTTTCGC | NO: 346 |
| | CGGACGTCAG | |
| proC-RA-SalI | CGTGCGGACGGCAAGCTGCTACAGCTGCGTGCGGTT | NO: 347 |
| | GGCCTGGCTGG | |
| LA-YZ-F | tgaattagaactcggtacgcgcgga | NO: 348 |
| LA-YZ-R | tagagaataggaacttcgaactgca | NO: 349 |
| RA-YZ-F | aacttcgaagcagctccagcctaca | NO: 350 |
| RA-YZ-R | atgattacgccaagtttgcacgcct | NO: 351 |
| proC-YZ-F | CAGCTTGCAGTCGGTTAACCAGGAC | NO: 352 |
| proC-YZ-R | cgccctgagtgcttgcggcagcgtg | NO: 353 |

Shake flask culture was conducted to verify spermidine yield of the strains E5-1, E5-2 and E5-3. Concentrations of spermidine in the supernatant in the shake flask were determined to be 19.3 g/L, 19.7 g/L and 21.3 g/L, respectively.

### EXAMPLE 12

### Knockout of Key Enzymes in Related Decomposition Pathways

Utilization and degradation pathways (competitive pathways) for Arginine (Arg): (1) Arg was degraded into N²-succinylarginine through arginine N-succinyltransferase encoded by the astA gene (with an amino acid sequence available under an NCBI accession number QJZ04124.1), and was further degraded into L-glutamate (L-Glu) and succinate through ast series of genes. To reduce the degradation of Arg, the Arg degradation pathway was weakened by deleting the astA gene.

Following the Red homologous recombination method described in Example 2, the astA gene was deleted based on the strain E5-3 as a parental strain for the metabolic pathway to prevent further degradation of Arg and redirect more metabolic flux toward spermidine synthesis. A strain E5-3ΔastA was constructed, which was designated as a strain E6. Primers used are shown in Table 16 below.

TABLE 16
Primer sequences (SEQ ID NO: 354-363)

| Primer name | Sequence 5′-3′ | SEQ |
|---|---|---|
| ApaI-LA-astA | CGCGCGGATCTTCCAGAGATTGGGCCCAC | NO: 354 |
| | GAACAGGCGAGTAAGTTC | |
| astA-LA-EcoRI | AAGCTTGACGTCCAGGTGCCTCTTAAGGA | NO: 355 |
| | TGAACCTCGGCTAACAAA | |
| KpnI-RA-astA | CACGCTAGCGGATCCGAGCTCGGTACCCT | NO: 356 |
| | TTATGGATTAACGGTGACT | |
| astA-RA-SalI | CGTGCGGACGGCAAGCTGCTACAGCTTTG | NO: 357 |
| | CTGCCATGCAGTAACCAC | |
| LA-YZ-F | tgaattagaactcggtacgcgcgga | NO: 358 |
| LA-YZ-R | tagagaataggaacttcgaactgca | NO: 359 |
| RA-YZ-F | aacttcgaagcagctccagcctaca | NO: 360 |
| RA-YZ-R | atgattacgccaagtttgcacgcct | NO: 361 |
| astA-YZ-F | GGGATCAGCAGGGGAAAGAG | NO: 362 |
| astA-YZ-R | cgccctgagtgcttgcggcagcgtg | NO: 363 |

Shake flask culture was conducted to verify spermidine yield of the strain E6. A concentration of spermidine in the supernatant in the shake flask was determined to be 24.7 g/L.

### EXAMPLE 13

### Comparison of Various Integrated Expression Strains and High-Cell Density Cultivation

The same procedures as in Examples 1-6 were further carried out in E. coli JM109, DH5α, Top 10, and MG1655, respectively, to obtain novel strains with an integrated synthetic pathway for spermidine production, which were designated as E.JM, E.DH, E.TO and E.MG. The obtained strains were subjected to high-cell density cultivation for 18 hours. The medium composition and fed-batch operation were subjected to the literature (Simple Fed-batch Technique for High Cell Density Cultivation of Escherichia coli, Journal of Biotechnology, 1995, 39:59-65). The resulting biomass and spermidine production are shown in Table 17.

The strains E.JM, E.DH, E.TO, and E.MG were also cultivated in shake flasks, and the spermidine yields were 19.7, 21.3, 22.7, and 26.2 g/L, respectively.

TABLE 17
Results of high-cell density cultivation of different strains

| Strain | Wet cell weight (g/L) | Spermidine concentration |
|---|---|---|
| E6 | 116 | 192 g/L |
| E.JM | 124 | 184 g/L |
| E.DH | 129 | 191 g/L |
| E.TO | 121 | 204 g/L |
| E.MG | 156 | 266 g/L |

Although the present disclosure has been disclosed as above in the form of preferred embodiments, it is not intended to limit the present disclosure. Those skilled in the art can make various modifications and variations without departing from the spirit and scope of the present disclosure. Therefore, the scope of protection of the present disclosure should be defined by the claims.

What is claimed is:

1. A genetically engineered strain, wherein the genetically engineered strain expresses exogenous carboxyaminopropylagmatine dehydrogenase (CAPADH), carboxyaminopropylagmatine decarboxylase (CAPADC), and aminopropylagmatine ureahydrolase (APAUH), and the genetically engineered strain is a genetically engineered strain of Serratia marcescens or a genetically engineered strain of Escherichia coli (E. coli); and the genetically engineered strain of E. coli is constructed using E. coli BL21 (DE3), E. coli JM109, E. coli DH5α, E. coli Top10, or E. coli MG1655 as hosts.

2. The genetically engineered strain of claim 1, wherein an argR gene encoding a repressor protein is knocked out from the genetically engineered strain, and an argA negative feedback mutant is constructed.

3. The genetically engineered strain of claim 1, wherein the genetically engineered strain utilizes a strong promoter to replace native promoters of a lysC gene encoding aspartate kinase, an asd gene encoding aspartate semialdehyde dehydrogenase, an argA gene encoding N-acetylglutamate synthase, an argH gene encoding arginine succinate lyase, and an speA gene encoding arginine decarboxylase.

4. The genetically engineered strain of claim 2, wherein the genetically engineered strain utilizes a strong promoter to replace native promoters of a lysC gene encoding aspartate kinase, an asd gene encoding aspartate semialdehyde dehydrogenase, an argA gene encoding N-acetylglutamate synthase, an argH gene encoding arginine succinate lyase, and an speA gene encoding arginine decarboxylase.

5. The genetically engineered strain of claim 1, wherein a gene potA encoding a transporter protein is knocked out from the genetically engineered strain; or the expression of transporter protein MdtJI that transports spermidine to an extracellular space is enhanced on the basis that the potA gene encoding a transporter protein is knocked out.

6. The genetically engineered strain of claim 1, wherein thrA and metL genes encoding homoserine synthase are further knocked out from the genetically engineered strain; or a dapA gene encoding dihydrodipicolinate synthase is further knocked out on the basis that the thrA and metL genes are knocked out; or a proC gene encoding pyrroline-5-carboxylate reductase is further knocked out on the basis of that the thrA, metL, and dapA genes are knocked out.

7. The genetically engineered strain of claim 6, wherein an astA gene encoding arginine N-succinyltransferase is knocked out from the genetically engineered strain.

8. The genetically engineered strain of claim 4, wherein the strong promoter is a trc promoter.


SEQUENCE LISTING (363 sequences)

SEQ ID NO: 1 - DNA, 39 bp, synthetic construct
  source 1..39: mol_type = other DNA
gcaccccaggcaagcttgatgggcacgggtgctcatcat

SEQ ID NO: 2 - DNA, 39 bp, synthetic construct
  source 1..39: mol_type = other DNA
gggctgcaggaattcgatatcttacgagaggcgccgctt

SEQ ID NO: 3 - DNA, 36 bp, synthetic construct
  source 1..36: mol_type = other DNA
cgatgatatcgaattcatgcgctatccccacttcct

SEQ ID NO: 4 - DNA, 35 bp, synthetic construct
  source 1..35: mol_type = other DNA
agttctagaactagtctaccgccggggtagatgcc

SEQ ID NO: 5 - DNA, 47 bp, synthetic construct
  source 1..47: mol_type = other DNA
cgcgcggatcttccagagattgggcccacctgccagagtggccgccg

SEQ ID NO: 6 - DNA, 45 bp, synthetic construct
  source 1..45: mol_type = other DNA
aagcttgacgtccaggtgcctcttaagtttgcaccgtggaatgtt

SEQ ID NO: 7 - DNA, 46 bp, synthetic construct
  source 1..46: mol_type = other DNA
cacgctagcggatccgagctcggtaccaacctggcgctaacaaagt

SEQ ID NO: 8 - DNA, 43 bp, synthetic construct
  source 1..43: mol_type = other DNA
cgtgcggacggcaagctgctacagctggatcacgcggtaggcg

SEQ ID NO: 9 - DNA, 25 bp, synthetic construct
  source 1..25: mol_type = other DNA
tgaattagaactcggtacgcgcgga

SEQ ID NO: 10 - DNA, 25 bp, synthetic construct
  source 1..25: mol_type = other DNA
tagagaataggaacttcgaactgca

SEQ ID NO: 11 - DNA, 25 bp, synthetic construct
  source 1..25: mol_type = other DNA
aacttcgaagcagctccagcctaca

SEQ ID NO: 12 - DNA, 25 bp, synthetic construct
  source 1..25: mol_type = other DNA
atgattacgccaagtttgcacgcct

SEQ ID NO: 13 - DNA, 22 bp, synthetic construct
  source 1..22: mol_type = other DNA
ttcagtgccggcgttctgaata

SEQ ID NO: 14 - DNA, 25 bp, synthetic construct
  source 1..25: mol_type = other DNA
cgccctgagtgcttgcggcagcgtg

SEQ ID NO: 15 - DNA, 23 bp, synthetic construct
  source 1..23: mol_type = other DNA
gtgaaggaacgtagtacagagct

SEQ ID NO: 16 - DNA, 19 bp, synthetic construct
  source 1..19: mol_type = other DNA
ttacagatccgccagcaga

SEQ ID NO: 17 - DNA, 21 bp, synthetic construct
  source 1..21: mol_type = other DNA
caaggattccgttactcagtc

SEQ ID NO: 18 - DNA, 21 bp, synthetic construct
  source 1..21: mol_type = other DNA
gacggaatccttgcaccagct

SEQ ID NO: 19 - DNA, 25 bp, synthetic construct
  source 1..25: mol_type = other DNA
tggaattcatgaaccaagcagcacc

SEQ ID NO: 20 - DNA, 33 bp, synthetic construct
  source 1..33: mol_type = other DNA
gccaagcttttattcgaacaggttgtgatgcag

SEQ ID NO: 21 - DNA, 25 bp, synthetic construct
  source 1..25: mol_type = other DNA
tcactgcataattcgtgtcgctcaa

SEQ ID NO: 22 - DNA, 25 bp, synthetic construct
  source 1..25: mol_type = other DNA
cgctactgccgccaggcaaattctg

SEQ ID NO: 23 - DNA, 41 bp, synthetic construct
  source 1..41: mol_type = other DNA
agcctacacgctagcggatccttgacaattaatcatccggc

SEQ ID NO: 24 - DNA, 45 bp, synthetic construct
  source 1..45: mol_type = other DNA
agtatctcaggtaccgagctcttactcaaacaaattactatgcag

SEQ ID NO: 25 - DNA, 25 bp, synthetic construct
  source 1..25: mol_type = other DNA
tcttctaataaggggatcttgaagt

SEQ ID NO: 26 - DNA, 25 bp, synthetic construct
  source 1..25: mol_type = other DNA
cgctgttgcggatcatctccagcgt

SEQ ID NO: 27 - DNA, 43 bp, synthetic construct
  source 1..43: mol_type = other DNA
cgcgcggatcttccagagattgggcccggcgcgctggctgcgg

SEQ ID NO: 28 - DNA, 46 bp, synthetic construct
  source 1..46: mol_type = other DNA
aagcttgacgtccaggtgcctcttaagctagaaacctcgtgtcagg

SEQ ID NO: 29 - DNA, 49 bp, synthetic construct
  source 1..49: mol_type = other DNA
cacgctagcggatccgagctcggtaccgtcattatccctgattgcgggc

SEQ ID NO: 30 - DNA, 45 bp, synthetic construct
  source 1..45: mol_type = other DNA
cgtgcggacggcaagctgctacagctggccagaccgagaacagcg

SEQ ID NO: 31 - DNA, 25 bp, synthetic construct
  source 1..25: mol_type = other DNA
tgaattagaactcggtacgcgcgga

SEQ ID NO: 32 - DNA, 25 bp, synthetic construct
  source 1..25: mol_type = other DNA
tagagaataggaacttcgaactgca

SEQ ID NO: 33 - DNA, 25 bp, synthetic construct
  source 1..25: mol_type = other DNA
aacttcgaagcagctccagcctaca

SEQ ID NO: 34 - DNA, 25 bp, synthetic construct
  source 1..25: mol_type = other DNA
atgattacgccaagtttgcacgcct

SEQ ID NO: 35 - DNA, 18 bp, synthetic construct
  source 1..18: mol_type = other DNA
tgcagcgcagcggctatc

SEQ ID NO: 36 - DNA, 25 bp, synthetic construct
  source 1..25: mol_type = other DNA
cgccctgagtgcttgcggcagcgtg

SEQ ID NO: 37 - DNA, 46 bp, synthetic construct
  source 1..46: mol_type = other DNA
aggaaacagaccatggaattcatgaaaaacgttggtttcatcggtt

SEQ ID NO: 38 - DNA, 39 bp, synthetic construct
  source 1..39: mol_type = other DNA
tccgccaaaacagccaagcttttacagcaggatgcgcag

SEQ ID NO: 39 - DNA, 25 bp, synthetic construct
  source 1..25: mol_type = other DNA
tcactgcataattcgtgtcgctcaa

SEQ ID NO: 40 - DNA, 25 bp, synthetic construct
  source 1..25: mol_type = other DNA
cgctactgccgccaggcaaattctg

SEQ ID NO: 41 - DNA, 41 bp, synthetic construct; identical to SEQ ID NO: 23
  source 1..41: mol_type = other DNA

SEQ ID NO: 42 - DNA, 42 bp, synthetic construct
  source 1..42: mol_type = other DNA
atcagtatctcaggtaccgagctcttacagcaggatgcgcag

SEQ ID NO: 43 - DNA, 25 bp, synthetic construct
  source 1..25: mol_type = other DNA
tcttctaataaggggatcttgaagt

SEQ ID NO: 44 - DNA, 25 bp, synthetic construct
  source 1..25: mol_type = other DNA
cgctgttgcggatcatctccagcgt

SEQ ID NO: 45 - DNA, 47 bp, synthetic construct
  source 1..47: mol_type = other DNA
cgcgcggatcttccagagattgggccccgaggagcgtctgaagcaac

SEQ ID NO: 46 - DNA, 46 bp, synthetic construct
  source 1..46: mol_type = other DNA
aagcttgacgtccaggtgcctcttaagaatatctgtgtcccgcccg

SEQ ID NO: 47 - DNA, 44 bp, synthetic construct
  source 1..44: mol_type = other DNA
cacgctagcggatccgagctcggtaccacccgcacttccagggg

SEQ ID NO: 48 - DNA, 41 bp, synthetic construct
  source 1..41: mol_type = other DNA
cgtgcggacggcaagctgctacagctgtggccggcctgcac

SEQ ID NO: 49 - DNA, 25 bp, synthetic construct
  source 1..25: mol_type = other DNA
tgaattagaactcggtacgcgcgga

SEQ ID NO: 50 - DNA, 25 bp, synthetic construct
  source 1..25: mol_type = other DNA
tagagaataggaacttcgaactgca

SEQ ID NO: 51 - DNA, 25 bp, synthetic construct
  source 1..25: mol_type = other DNA
aacttcgaagcagctccagcctaca

SEQ ID NO: 52 - DNA, 25 bp, synthetic construct
  source 1..25: mol_type = other DNA
atgattacgccaagtttgcacgcct

SEQ ID NO: 53 - DNA, 15 bp, synthetic construct
  source 1..15: mol_type = other DNA
gccatgcagcgcacc

SEQ ID NO: 54 - DNA, 25 bp, synthetic construct
  source 1..25: mol_type = other DNA
cgccctgagtgcttgcggcagcgtg

SEQ ID NO: 55 - DNA, 41 bp, synthetic construct
  source 1..41: mol_type = other DNA
aggaaacagaccatggaattcgtgaaggaacgtagtacaga

SEQ ID NO: 56 - DNA, 43 bp, synthetic construct
  source 1..43: mol_type = other DNA
tccgccaaaacagccaagcttttacagatcggccagcaggatt

SEQ ID NO: 57 - DNA, 25 bp, synthetic construct
  source 1..25: mol_type = other DNA
tcactgcataattcgtgtcgctcaa

SEQ ID NO: 58 - DNA, 25 bp, synthetic construct
  source 1..25: mol_type = other DNA
cgctactgccgccaggcaaattctg

SEQ ID NO: 59 - DNA, 41 bp, synthetic construct; identical to SEQ ID NO: 23
  source 1..41: mol_type = other DNA

SEQ ID NO: 60 - DNA, 46 bp, synthetic construct
  source 1..46: mol_type = other DNA
atcagtatctcaggtaccgagctcttacagatcggccagcaggatt

SEQ ID NO: 61 - DNA, 25 bp, synthetic construct
  source 1..25: mol_type = other DNA
tcttctaataaggggatcttgaagt

SEQ ID NO: 62 - DNA, 25 bp, synthetic construct
  source 1..25: mol_type = other DNA
cgctgttgcggatcatctccagcgt

SEQ ID NO: 63 - DNA, 44 bp, synthetic construct
  source 1..44: mol_type = other DNA
cgcgcggatcttccagagattgggcccgccttggcgacgcgcac

SEQ ID NO: 64 - DNA, 48 bp, synthetic construct
  source 1..48: mol_type = other DNA
aagcttgacgtccaggtgcctcttaagggccaaacctctttgcatatt

SEQ ID NO: 65 - DNA, 42 bp, synthetic construct
  source 1..42: mol_type = other DNA
cacgctagcggatccgagctcggtacctctccgcggcgcggc

SEQ ID NO: 66 - DNA, 47 bp, synthetic construct
  source 1..47: mol_type = other DNA
cgtgcggacggcaagctgctacagctggtgattttcctcggcgatcg

SEQ ID NO: 67 - DNA, 25 bp, synthetic construct
  source 1..25: mol_type = other DNA
tgaattagaactcggtacgcgcgga

SEQ ID NO: 68 - DNA, 25 bp, synthetic construct
  source 1..25: mol_type = other DNA
tagagaataggaacttcgaactgca

SEQ ID NO: 69 - DNA, 25 bp, synthetic construct
  source 1..25: mol_type = other DNA
aacttcgaagcagctccagcctaca

SEQ ID NO: 70 - DNA, 25 bp, synthetic construct
  source 1..25: mol_type = other DNA
atgattacgccaagtttgcacgcct

SEQ ID NO: 71 - DNA, 21 bp, synthetic construct
  source 1..21: mol_type = other DNA
gcctttggtggacaatgcgaa

SEQ ID NO: 72 - DNA, 25 bp, synthetic construct
  source 1..25: mol_type = other DNA
cgccctgagtgcttgcggcagcgtg

SEQ ID NO: 73 - DNA, 38 bp, synthetic construct
  source 1..38: mol_type = other DNA
aggaaacagaccatggaattcatggcactttggggcgg

SEQ ID NO: 74 - DNA, 37 bp, synthetic construct
  source 1..37: mol_type = other DNA
tccgccaaaacagccaagctttcaagccaaacgctgt

SEQ ID NO: 75 - DNA, 25 bp, synthetic construct
  source 1..25: mol_type = other DNA
tcactgcataattcgtgtcgctcaa

SEQ ID NO: 76 - DNA, 25 bp, synthetic construct
  source 1..25: mol_type = other DNA
cgctactgccgccaggcaaattctg

SEQ ID NO: 77 - DNA, 41 bp, synthetic construct; identical to SEQ ID NO: 23
  source 1..41: mol_type = other DNA

SEQ ID NO: 78 - DNA, 40 bp, synthetic construct
  source 1..40: mol_type = other DNA
atcagtatctcaggtaccgagctctcaagccaaacgctgt

SEQ ID NO: 79 - DNA, 25 bp, synthetic construct
  source 1..25: mol_type = other DNA
tcttctaataaggggatcttgaagt

SEQ ID NO: 80 - DNA, 25 bp, synthetic construct
  source 1..25: mol_type = other DNA
cgctgttgcggatcatctccagcgt

SEQ ID NO: 81 - DNA, 43 bp, synthetic construct
  source 1..43: mol_type = other DNA
cgcgcggatcttccagagattgggcccaatggcgcggccgatc

SEQ ID NO: 82 - DNA, 47 bp, synthetic construct
  source 1..47: mol_type = other DNA
aagcttgacgtccaggtgcctcttaagacctttactcctgaattttt

SEQ ID NO: 83 - DNA, 42 bp, synthetic construct
  source 1..42: mol_type = other DNA
cacgctagcggatccgagctcggtacccgacgacaagggcgg

SEQ ID NO: 84 - DNA, 42 bp, synthetic construct
  source 1..42: mol_type = other DNA
cgtgcggacggcaagctgctacagctgatgccggcagcttcc

SEQ ID NO: 85 - DNA, 25 bp, synthetic construct
  source 1..25: mol_type = other DNA
tgaattagaactcggtacgcgcgga

SEQ ID NO: 86 - DNA, 25 bp, synthetic construct
  source 1..25: mol_type = other DNA
tagagaataggaacttcgaactgca

SEQ ID NO: 87 - DNA, 25 bp, synthetic construct
  source 1..25: mol_type = other DNA
aacttcgaagcagctccagcctaca

SEQ ID NO: 88 - DNA, 25 bp, synthetic construct
  source 1..25: mol_type = other DNA
atgattacgccaagtttgcacgcct

SEQ ID NO: 89 - DNA, 18 bp, synthetic construct
  source 1..18: mol_type = other DNA
agcgcagcgatctggaag

SEQ ID NO: 90 - DNA, 25 bp, synthetic construct
  source 1..25: mol_type = other DNA
cgccctgagtgcttgcggcagcgtg

SEQ ID NO: 91 - DNA, 40 bp, synthetic construct
  source 1..40: mol_type = other DNA
aggaaacagaccatggaattcatgtctgatgatctgttga

SEQ ID NO: 92 - DNA, 44 bp, synthetic construct
  source 1..44: mol_type = other DNA
tccgccaaaacagccaagcttttactcgtcttccagatacgtgt

SEQ ID NO: 93 - DNA, 25 bp, synthetic construct
  source 1..25: mol_type = other DNA
tcactgcataattcgtgtcgctcaa

SEQ ID NO: 94 - DNA, 25 bp, synthetic construct
  source 1..25: mol_type = other DNA
cgctactgccgccaggcaaattctg

SEQ ID NO: 95 - DNA, 41 bp, synthetic construct; identical to SEQ ID NO: 23
  source 1..41: mol_type = other DNA

SEQ ID NO: 96 - DNA, 47 bp, synthetic construct
  source 1..47: mol_type = other DNA
atcagtatctcaggtaccgagctcttactcgtcttccagatacgtgt

SEQ ID NO: 97 - DNA, 25 bp, synthetic construct
  source 1..25: mol_type = other DNA
tcttctaataaggggatcttgaagt

SEQ ID NO: 98 - DNA, 25 bp, synthetic construct
  source 1..25: mol_type = other DNA
cgctgttgcggatcatctccagcgt

SEQ ID NO: 99 - DNA, 42 bp, synthetic construct
  source 1..42: mol_type = other DNA
cgcgcggatcttccagagattgggcccatggcgtcgaggacg

SEQ ID NO: 100 - DNA, 45 bp, synthetic construct
  source 1..45: mol_type = other DNA
aagcttgacgtccaggtgcctcttaagggttcgcctcacattccg

SEQ ID NO: 101 - DNA, 43 bp, synthetic construct
  source 1..43: mol_type = other DNA
cacgctagcggatccgagctcggtaccccgccctcttcagggg

SEQ ID NO: 102 - DNA, 46 bp, synthetic construct
  source 1..46: mol_type = other DNA
cgtgcggacggcaagctgctacagctgccaacgatgttcagcgact

SEQ ID NO: 103 - DNA, 25 bp, synthetic construct
  source 1..25: mol_type = other DNA
tgaattagaactcggtacgcgcgga

SEQ ID NO: 104 - DNA, 25 bp, synthetic construct
  source 1..25: mol_type = other DNA
tagagaataggaacttcgaactgca

SEQ ID NO: 105 - DNA, 25 bp, synthetic construct
  source 1..25: mol_type = other DNA
aacttcgaagcagctccagcctaca

SEQ ID NO: 106 - DNA, 25 bp, synthetic construct
  source 1..25: mol_type = other DNA
atgattacgccaagtttgcacgcct

SEQ ID NO: 107 - DNA, 16 bp, synthetic construct
  source 1..16: mol_type = other DNA
atcgacccaggcgctg

SEQ ID NO: 108 - DNA, 25 bp, synthetic construct
  source 1..25: mol_type = other DNA
cgccctgagtgcttgcggcagcgtg

SEQ ID NO: 109 - DNA, 47 bp, synthetic construct
  source 1..47: mol_type = other DNA
cgcgcggatcttccagagattgggcccggctttcatccgccggaact

SEQ ID NO: 110 - DNA, 47 bp, synthetic construct
  source 1..47: mol_type = other DNA
aagcttgacgtccaggtgcctcttaaggtaaacgcaacggatggctt

SEQ ID NO: 111 - DNA, 47 bp, synthetic construct
  source 1..47: mol_type = other DNA
cacgctagcggatccgagctcggtaccgttccagaatgtagtgattg

SEQ ID NO: 112 - DNA, 47 bp, synthetic construct
  source 1..47: mol_type = other DNA
cgtgcggacggcaagctgctacagctgtgtcgttgttcatcagcagg

SEQ ID NO: 113 - DNA, 25 bp, synthetic construct
  source 1..25: mol_type = other DNA
tgaattagaactcggtacgcgcgga

SEQ ID NO: 114 - DNA, 25 bp, synthetic construct
  source 1..25: mol_type = other DNA
tagagaataggaacttcgaactgca

SEQ ID NO: 115 - DNA, 25 bp, synthetic construct
  source 1..25: mol_type = other DNA
aacttcgaagcagctccagcctaca

SEQ ID NO: 116 - DNA, 25 bp, synthetic construct
  source 1..25: mol_type = other DNA
atgattacgccaagtttgcacgcct

SEQ ID NO: 117 - DNA, 25 bp, synthetic construct
  source 1..25: mol_type = other DNA
cggtcgaaatcacggatgatgtagt

SEQ ID NO: 118 - DNA, 25 bp, synthetic construct
  source 1..25: mol_type = other DNA
cgccctgagtgcttgcggcagcgtg

SEQ ID NO: 119 - DNA, 46 bp, synthetic construct
  source 1..46: mol_type = other DNA
aggaaacagaccatggaattcatgatttattggatctttttaggtt

SEQ ID NO: 120 - DNA, 41 bp, synthetic construct
  source 1..41: mol_type = other DNA
tccgccaaaacagccaagctttcaaactgttgcatggttgt

SEQ ID NO: 121 - DNA, 25 bp, synthetic construct
  source 1..25: mol_type = other DNA
tcactgcataattcgtgtcgctcaa

SEQ ID NO: 122 - DNA, 25 bp, synthetic construct
  source 1..25: mol_type = other DNA
cgctactgccgccaggcaaattctg

SEQ ID NO: 123 - DNA, 41 bp, synthetic construct; identical to SEQ ID NO: 23
  source 1..41: mol_type = other DNA

SEQ ID NO: 124 - DNA, 41 bp, synthetic construct
  source 1..41: mol_type = other DNA
agtatctcaggtaccgagctctcaaactgttgcatggttgt

SEQ ID NO: 125 - DNA, 25 bp, synthetic construct
  source 1..25: mol_type = other DNA
tcttctaataaggggatcttgaagt

SEQ ID NO: 126 - DNA, 25 bp, synthetic construct
  source 1..25: mol_type = other DNA
cgctgttgcggatcatctccagcgt

SEQ ID NO: 127 - DNA, 44 bp, synthetic construct
  source 1..44: mol_type = other DNA
cgcgcggatcttccagagattgggcccggaggcgatcatcggcg

SEQ ID NO: 128 - DNA, 44 bp, synthetic construct
  source 1..44: mol_type = other DNA
aagcttgacgtccaggtgcctcttaagaatttttctcccaaacg

SEQ ID NO: 129 - DNA, 46 bp, synthetic construct
  source 1..46: mol_type = other DNA
cacgctagcggatccgagctcggtacctagcgggcaataaaaaacc

SEQ ID NO: 130 - DNA, 45 bp, synthetic construct
  source 1..45: mol_type = other DNA
cgtgcggacggcaagctgctacagctgtcttccgccacttcgctg

SEQ ID NO: 131 - DNA, 25 bp, synthetic construct
  source 1..25: mol_type = other DNA
tgaattagaactcggtacgcgcgga

SEQ ID NO: 132 - DNA, 25 bp, synthetic construct
  source 1..25: mol_type = other DNA
tagagaataggaacttcgaactgca

SEQ ID NO: 133 - DNA, 25 bp, synthetic construct
  source 1..25: mol_type = other DNA
aacttcgaagcagctccagcctaca

SEQ ID NO: 134 - DNA, 25 bp, synthetic construct
  source 1..25: mol_type = other DNA
atgattacgccaagtttgcacgcct

SEQ ID NO: 135 - DNA, 19 bp, synthetic construct
  source 1..19: mol_type = other DNA
gcggtgccgctggcgaaag

SEQ ID NO: 136 - DNA, 25 bp, synthetic construct
  source 1..25: mol_type = other DNA
cgccctgagtgcttgcggcagcgtg

SEQ ID NO: 137 - DNA, 43 bp, synthetic construct
  source 1..43: mol_type = other DNA
cgcgcggatcttccagagattgggcccaaaatgtcggcgcggc

SEQ ID NO: 138 - DNA, 48 bp, synthetic construct
  source 1..48: mol_type = other DNA
aagcttgacgtccaggtgcctcttaaggtgtgatctctccgaatttaa

SEQ ID NO: 139 - DNA, 48 bp, synthetic construct
  source 1..48: mol_type = other DNA
cacgctagcggatccgagctcggtacctatggttaaggtgtatgcacc

SEQ ID NO: 140 - DNA, 46 bp, synthetic construct
  source 1..46: mol_type = other DNA
cgtgcggacggcaagctgctacagctgccttgtttgatcgcctgcg

SEQ ID NO: 141 - DNA, 25 bp, synthetic construct
  source 1..25: mol_type = other DNA
tgaattagaactcggtacgcgcgga

SEQ ID NO: 142 - DNA, 25 bp, synthetic construct
  source 1..25: mol_type = other DNA
tagagaataggaacttcgaactgca

SEQ ID NO: 143 - DNA, 25 bp, synthetic construct
  source 1..25: mol_type = other DNA
aacttcgaagcagctccagcctaca

SEQ ID NO: 144 - DNA, 25 bp, synthetic construct
  source 1..25: mol_type = other DNA
atgattacgccaagtttgcacgcct

SEQ ID NO: 145 - DNA, 20 bp, synthetic construct
  source 1..20: mol_type = other DNA
acataagcgcgaataatgac

SEQ ID NO: 146 - DNA, 25 bp, synthetic construct
  source 1..25: mol_type = other DNA
cgccctgagtgcttgcggcagcgtg

SEQ ID NO: 147 - DNA, 42 bp, synthetic construct
  source 1..42: mol_type = other DNA
cgcgcggatcttccagagattgggcccacgtagtgcagcgcg

SEQ ID NO: 148 - DNA, 44 bp, synthetic construct
  source 1..44: mol_type = other DNA
aagcttgacgtccaggtgcctcttaaggcttacctcgttgccgc

SEQ ID NO: 149 - DNA, 42 bp, synthetic construct
  source 1..42: mol_type = other DNA
cacgctagcggatccgagctcggtacctcctctgcgggcgcg

SEQ ID NO: 150 - DNA, 45 bp, synthetic construct
  source 1..45: mol_type = other DNA
cgtgcggacggcaagctgctacagctgtccatggcgatgttggcg

SEQ ID NO: 151 - DNA, 25 bp, synthetic construct
  source 1..25: mol_type = other DNA
tgaattagaactcggtacgcgcgga

SEQ ID NO: 152 - DNA, 25 bp, synthetic construct
  source 1..25: mol_type = other DNA
tagagaataggaacttcgaactgca

SEQ ID NO: 153 - DNA, 25 bp, synthetic construct
  source 1..25: mol_type = other DNA
aacttcgaagcagctccagcctaca

SEQ ID NO: 154 - DNA, 25 bp, synthetic construct
  source 1..25: mol_type = other DNA
atgattacgccaagtttgcacgcct

SEQ ID NO: 155 - DNA, 18 bp, synthetic construct
  source 1..18: mol_type = other DNA
gaacgacgacgaacagta

SEQ ID NO: 156 - DNA, 25 bp, synthetic construct
  source 1..25: mol_type = other DNA
cgccctgagtgcttgcggcagcgtg

SEQ ID NO: 157 - DNA, 42 bp, synthetic construct
  source 1..42: mol_type = other DNA
cgcgcggatcttccagagattgggcccaacgttggcccgcag

SEQ ID NO: 158 - DNA, 47 bp, synthetic construct
  source 1..47: mol_type = other DNA
aagcttgacgtccaggtgcctcttaagcaaatccctcgtctgtgtgt

SEQ ID NO: 159 - DNA, 49 bp, synthetic construct
  source 1..49: mol_type = other DNA
cacgctagcggatccgagctcggtaccataaccccgagttaaggagaag

SEQ ID NO: 160 - DNA, 43 bp, synthetic construct
  source 1..43: mol_type = other DNA
cgtgcggacggcaagctgctacagctgtccacgcccagatcgg

SEQ ID NO: 161 - DNA, 25 bp, synthetic construct
  source 1..25: mol_type = other DNA
tgaattagaactcggtacgcgcgga

SEQ ID NO: 162 - DNA, 25 bp, synthetic construct
  source 1..25: mol_type = other DNA
tagagaataggaacttcgaactgca

SEQ ID NO: 163 - DNA, 25 bp, synthetic construct
  source 1..25: mol_type = other DNA
aacttcgaagcagctccagcctaca

SEQ ID NO: 164 - DNA, 25 bp, synthetic construct
  source 1..25: mol_type = other DNA
atgattacgccaagtttgcacgcct

SEQ ID NO: 165 - DNA, 17 bp, synthetic construct
  source 1..17: mol_type = other DNA
ccagcgtggtggacttg

SEQ ID NO: 166 - DNA, 47 bp, synthetic construct
  source 1..47: mol_type = other DNA
cgcgcggatcttccagagattgggcccacgaacaggcgagtaagttc

SEQ ID NO: 167 - DNA, 46 bp, synthetic construct
  source 1..46: mol_type = other DNA
cgcgcggatcttccagagattgggcccgaagatcctgcaggatgcc

SEQ ID NO: 168 - DNA, 45 bp, synthetic construct
  source 1..45: mol_type = other DNA
aagcttgacgtccaggtgcctcttaagaaaatattccttctgtgt

SEQ ID NO: 169 - DNA, 45 bp, synthetic construct
  source 1..45: mol_type = other DNA
cacgctagcggatccgagctcggtaccatttgttagctaagccgc

SEQ ID NO: 170 - DNA, 42 bp, synthetic construct
  source 1..42: mol_type = other DNA
cgtgcggacggcaagctgctacagctgcggaggaggcctggc

SEQ ID NO: 171 - DNA, 25 bp, synthetic construct
  source 1..25: mol_type = other DNA
tgaattagaactcggtacgcgcgga

SEQ ID NO: 172 - DNA, 25 bp, synthetic construct
  source 1..25: mol_type = other DNA
tagagaataggaacttcgaactgca

SEQ ID NO: 173 - DNA, 25 bp, synthetic construct
  source 1..25: mol_type = other DNA
aacttcgaagcagctccagcctaca

SEQ ID NO: 174 - DNA, 25 bp, synthetic construct
  source 1..25: mol_type = other DNA
atgattacgccaagtttgcacgcct

SEQ ID NO: 175 - DNA, 17 bp, synthetic construct
  source 1..17: mol_type = other DNA
ggcgcattctctgcgcg

SEQ ID NO: 176 - DNA, 25 bp, synthetic construct
  source 1..25: mol_type = other DNA
cgccctgagtgcttgcggcagcgtg

SEQ ID NO: 177 - DNA, 46 bp, synthetic construct
  source 1..46: mol_type = other DNA
cgcgcggatcttccagagattgggcccggccaaaaaactgatcgac

SEQ ID NO: 178 - DNA, 49 bp, synthetic construct
  source 1..49: mol_type = other DNA
aagcttgacgtccaggtgcctcttaagaatgcgaacctcttctgaatca

SEQ ID NO: 179 - DNA, 47 bp, synthetic construct
  source 1..47: mol_type = other DNA
cacgctagcggatccgagctcggtaccgtgctcccccttattgcaca

SEQ ID NO: 180 - DNA, 43 bp, synthetic construct
  source 1..43: mol_type = other DNA
cgtgcggacggcaagctgctacagctaccaccccgcccatgaa

SEQ ID NO: 181 - DNA, 25 bp, synthetic construct
  source 1..25: mol_type = other DNA
tgaattagaactcggtacgcgcgga

SEQ ID NO: 182 - DNA, 25 bp, synthetic construct
  source 1..25: mol_type = other DNA
tagagaataggaacttcgaactgca

SEQ ID NO: 183 - DNA, 25 bp, synthetic construct
  source 1..25: mol_type = other DNA
aacttcgaagcagctccagcctaca

SEQ ID NO: 184 - DNA, 25 bp, synthetic construct
  source 1..25: mol_type = other DNA
atgattacgccaagtttgcacgcct

SEQ ID NO: 185 - DNA, 18 bp, synthetic construct
  source 1..18: mol_type = other DNA
gtcccggtttatgcccct

SEQ ID NO: 186 - DNA, 25 bp, synthetic construct
  source 1..25: mol_type = other DNA
cgccctgagtgcttgcggcagcgtg

SEQ ID NO: 187 - DNA, 30 bp, synthetic construct
  source 1..30: mol_type = other DNA
ttgacaattaatcatccggctcgtataatg

SEQ ID NO: 188 - DNA, 37 bp, synthetic construct
  source 1..37: mol_type = other DNA
ccacagccaggatccgatgggcacgggtgctcatcat

SEQ ID NO: 189 - DNA, 39 bp, synthetic construct
  source 1..39: mol_type = other DNA
gcattatgcggccgcaagcttttacgagaggcgccgctt

SEQ ID NO: 190 - DNA, 41 bp, synthetic construct
  source 1..41: mol_type = other DNA
gcgatcgctgacgtcggtaccatgcgctatccccacttcct

SEQ ID NO: 191 - DNA, 41 bp, synthetic construct
  source 1..41: mol_type = other DNA
ggtttctttaccagactcgagctaccgccggggtagatgcc

SEQ ID NO: 192 - DNA, 47 bp, synthetic construct
  source 1..47: mol_type = other DNA
cgcgcggatcttccagagattgggcccacctgtgacagcagcggcag

SEQ ID NO: 193 - DNA, 47 bp, synthetic construct
  source 1..47: mol_type = other DNA
aagcttgacgtccaggtgcctcttaagaagtcacccgatatggtggt

SEQ ID NO: 194 - DNA, 47 bp, synthetic construct
  source 1..47: mol_type = other DNA
cacgctagcggatccgagctcggtacctctctgccccgtcgtttctg

SEQ ID NO: 195 - DNA, 47 bp, synthetic construct
  source 1..47: mol_type = other DNA
cgtgcggacggcaagctgctacagctgcgatgcacggcagaactcgc

SEQ ID NO: 196 - DNA, 25 bp, synthetic construct
  source 1..25: mol_type = other DNA
tgaattagaactcggtacgcgcgga

SEQ ID NO: 197 - DNA, 25 bp, synthetic construct
  source 1..25: mol_type = other DNA
tagagaataggaacttcgaactgca

SEQ ID NO: 198 - DNA, 25 bp, synthetic construct
  source 1..25: mol_type = other DNA
aacttcgaagcagctccagcctaca

SEQ ID NO: 199 - DNA, 25 bp, synthetic construct
  source 1..25: mol_type = other DNA
atgattacgccaagtttgcacgcct

SEQ ID NO: 200 - DNA, 25 bp, synthetic construct
  source 1..25: mol_type = other DNA
agacccgccaccggccttcgcttca

SEQ ID NO: 201 - DNA, 25 bp, synthetic construct
  source 1..25: mol_type = other DNA
cgccctgagtgcttgcggcagcgtg

SEQ ID NO: 202 - DNA, 23 bp, synthetic construct
  source 1..23: mol_type = other DNA
gtggtaaaggaacgtaaaaccga

SEQ ID NO: 203 - DNA, 24 bp, synthetic construct
  source 1..24: mol_type = other DNA
ttaccctaaatccgccatcaacac

SEQ ID NO: 204 - DNA, 26 bp, synthetic construct
  source 1..26: mol_type = other DNA
gtcgagggattccgctattcggttcc

SEQ ID NO: 205 - DNA, 25 bp, synthetic construct
  source 1..25: mol_type = other DNA
agcggaatccctcgaccaactcggt

SEQ ID NO: 206 - DNA, 30 bp, synthetic construct
  source 1..30: mol_type = other DNA
tggaattcatgtctgaaattgttgtctcca

SEQ ID NO: 207 - DNA, 33 bp, synthetic construct
  source 1..33: mol_type = other DNA
gccaagcttttactcaaacaaattactatgcag

SEQ ID NO: 208 - DNA, 25 bp, synthetic construct
  source 1..25: mol_type = other DNA
tcactgcataattcgtgtcgctcaa

SEQ ID NO: 209 - DNA, 25 bp, synthetic construct
  source 1..25: mol_type = other DNA
cgctactgccgccaggcaaattctg

SEQ ID NO: 210 - DNA, 41 bp, synthetic construct; identical to SEQ ID NO: 23
  source 1..41: mol_type = other DNA

SEQ ID NO: 211 - DNA, 45 bp, synthetic construct; identical to SEQ ID NO: 24
  source 1..45: mol_type = other DNA

SEQ ID NO: 212 - DNA, 25 bp, synthetic construct
  source 1..25: mol_type = other DNA
tcttctaataaggggatcttgaagt

SEQ ID NO: 213 - DNA, 25 bp, synthetic construct
  source 1..25: mol_type = other DNA
cgctgttgcggatcatctccagcgt

SEQ ID NO: 214 - DNA, 46 bp, synthetic construct
  source 1..46: mol_type = other DNA
cgcgcggatcttccagagattgggccctcagaaccgccgatcccga

SEQ ID NO: 215 - DNA, 47 bp, synthetic construct
  source 1..47: mol_type = other DNA
aagcttgacgtccaggtgcctcttaagaactacctcgtgtcagggga

SEQ ID NO: 216 - DNA, 47 bp, synthetic construct
  source 1..47: mol_type = other DNA
cacgctagcggatccgagctcggtaccatactgtatggcctggaagc

SEQ ID NO: 217 - DNA, 46 bp, synthetic construct
  source 1..46: mol_type = other DNA
cgtgcggacggcaagctgctacagctgccgcattaccctggtgcag

SEQ ID NO: 218 - DNA, 25 bp, synthetic construct
  source 1..25: mol_type = other DNA
tgaattagaactcggtacgcgcgga

SEQ ID NO: 219 - DNA, 25 bp, synthetic construct
  source 1..25: mol_type = other DNA
tagagaataggaacttcgaactgca

SEQ ID NO: 220 - DNA, 25 bp, synthetic construct
  source 1..25: mol_type = other DNA
aacttcgaagcagctccagcctaca

SEQ ID NO: 221 - DNA, 25 bp, synthetic construct
  source 1..25: mol_type = other DNA
atgattacgccaagtttgcacgcct

SEQ ID NO: 222 - DNA, 20 bp, synthetic construct
  source 1..20: mol_type = other DNA
tcagaaccgccgatcccgat

SEQ ID NO: 223 - DNA, 25 bp, synthetic construct
  source 1..25: mol_type = other DNA
cgccctgagtgcttgcggcagcgtg

SEQ ID NO: 224 - DNA, 41 bp, synthetic construct
  source 1..41: mol_type = other DNA
aggaaacagaccatggaattcatgaaaaatgttggttttat

SEQ ID NO: 225 - DNA, 41 bp, synthetic construct
  source 1..41: mol_type = other DNA
tccgccaaaacagccaagcttttacgccagttgacgaagca

SEQ ID NO: 226 - DNA, 25 bp, synthetic construct
  source 1..25: mol_type = other DNA
tcactgcataattcgtgtcgctcaa

SEQ ID NO: 227 - DNA, 25 bp, synthetic construct
  source 1..25: mol_type = other DNA
cgctactgccgccaggcaaattctg

SEQ ID NO: 228 - DNA, 41 bp, synthetic construct; identical to SEQ ID NO: 23
  source 1..41: mol_type = other DNA

SEQ ID NO: 229 - DNA, 44 bp, synthetic construct
  source 1..44: mol_type = other DNA
atcagtatctcaggtaccgagctcttacgccagttgacgaagca

SEQ ID NO: 230 - DNA, 25 bp, synthetic construct
  source 1..25: mol_type = other DNA
tcttctaataaggggatcttgaagt

SEQ ID NO: 231 - DNA, 25 bp, synthetic construct
  source 1..25: mol_type = other DNA
cgctgttgcggatcatctccagcgt

SEQ ID NO: 232 - DNA, 47 bp, synthetic construct
  source 1..47: mol_type = other DNA
cgcgcggatcttccagagattgggcccttgttgtcagccacgttaac

SEQ ID NO: 233 - DNA, 47 bp, synthetic construct
  source 1..47: mol_type = other DNA
aagcttgacgtccaggtgcctcttaagaagcgtttttttcctgcaaa

SEQ ID NO: 234 - DNA, 48 bp, synthetic construct
  source 1..48: mol_type = other DNA
cacgctagcggatccgagctcggtacctctttattcattaaatctggg

SEQ ID NO: 235 - DNA, 47 bp, synthetic construct
  source 1..47: mol_type = other DNA
cgtgcggacggcaagctgctacagctgattggcgcatcaaactgatt

SEQ ID NO: 236 - DNA, 25 bp, synthetic construct
  source 1..25: mol_type = other DNA
tgaattagaactcggtacgcgcgga

SEQ ID NO: 237 - DNA, 25 bp, synthetic construct
  source 1..25: mol_type = other DNA
tagagaataggaacttcgaactgca

SEQ ID NO: 238 - DNA, 25 bp, synthetic construct
  source 1..25: mol_type = other DNA
aacttcgaagcagctccagcctaca

SEQ ID NO: 239 - DNA, 25 bp, synthetic construct
  source 1..25: mol_type = other DNA
atgattacgccaagtttgcacgcct

SEQ ID NO: 240 - DNA, 20 bp, synthetic construct
  source 1..20: mol_type = other DNA
ttgttgtcagccacgttaac

SEQ ID NO: 241 - DNA, 25 bp, synthetic construct
  source 1..25: mol_type = other DNA
cgccctgagtgcttgcggcagcgtg

SEQ ID NO: 242 - DNA, 41 bp, synthetic construct
  source 1..41: mol_type = other DNA
aggaaacagaccatggaattcgtggtaaaggaacgtaaaac

SEQ ID NO: 243 - DNA, 41 bp, synthetic construct
  source 1..41: mol_type = other DNA
tccgccaaaacagccaagcttttaccctaaatccgccatca

SEQ ID NO: 244 - DNA, 25 bp, synthetic construct
  source 1..25: mol_type = other DNA
tcactgcataattcgtgtcgctcaa

SEQ ID NO: 245 - DNA, 25 bp, synthetic construct
  source 1..25: mol_type = other DNA
cgctactgccgccaggcaaattctg

SEQ ID NO: 246 - DNA, 41 bp, synthetic construct; identical to SEQ ID NO: 23
  source 1..41: mol_type = other DNA

SEQ ID NO: 247 - DNA, 44 bp, synthetic construct
  source 1..44: mol_type = other DNA
atcagtatctcaggtaccgagctcttaccctaaatccgccatca

SEQ ID NO: 248 - DNA, 25 bp, synthetic construct
  source 1..25: mol_type = other DNA
tcttctaataaggggatcttgaagt

SEQ ID NO: 249 - DNA, 25 bp, synthetic construct
  source 1..25: mol_type = other DNA
cgctgttgcggatcatctccagcgt

SEQ ID NO: 250 - DNA, 47 bp, synthetic construct
  source 1..47: mol_type = other DNA
cgcgcggatcttccagagattgggccctacgcgcacttgcaatggaa

SEQ ID NO: 251 - DNA, 47 bp, synthetic construct
  source 1..47: mol_type = other DNA
aagcttgacgtccaggtgcctcttaagggcacacctctttgcatgat

SEQ ID NO: 252 - DNA, 47 bp, synthetic construct
  source 1..47: mol_type = other DNA
cacgctagcggatccgagctcggtaccgcgatgaaaatcgtcggatg

SEQ ID NO: 253 - DNA, 47 bp, synthetic construct
  source 1..47: mol_type = other DNA
cgtgcggacggcaagctgctacagctggatcgtgatcaactggcctc

SEQ ID NO: 254 - DNA, 25 bp, synthetic construct
  source 1..25: mol_type = other DNA
tgaattagaactcggtacgcgcgga

SEQ ID NO: 255 - DNA, 25 bp, synthetic construct
  source 1..25: mol_type = other DNA
tagagaataggaacttcgaactgca

SEQ ID NO: 256 - DNA, 25 bp, synthetic construct
  source 1..25: mol_type = other DNA
aacttcgaagcagctccagcctaca

SEQ ID NO: 257 - DNA, 25 bp, synthetic construct
  source 1..25: mol_type = other DNA
atgattacgccaagtttgcacgcct

SEQ ID NO: 258 - DNA, 20 bp, synthetic construct
  source 1..20: mol_type = other DNA
gctgtttctgggcttttgct

SEQ ID NO: 259 - DNA, 25 bp, synthetic construct
  source 1..25: mol_type = other DNA
cgccctgagtgcttgcggcagcgtg

SEQ ID NO: 260 - DNA, 41 bp, synthetic construct
  source 1..41: mol_type = other DNA
aggaaacagaccatggaattcatggcactttggggcgggcg

SEQ ID NO: 261 - DNA, 41 bp, synthetic construct
  source 1..41: mol_type = other DNA
tccgccaaaacagccaagcttttaccctaaccgagcctgcg

SEQ ID NO: 262 - DNA, 25 bp, synthetic construct
  source 1..25: mol_type = other DNA
tcactgcataattcgtgtcgctcaa

SEQ ID NO: 263 - DNA, 25 bp, synthetic construct
  source 1..25: mol_type = other DNA
cgctactgccgccaggcaaattctg

SEQ ID NO: 264 - DNA, 41 bp, synthetic construct; identical to SEQ ID NO: 23
  source 1..41: mol_type = other DNA

SEQ ID NO: 265 - DNA, 44 bp, synthetic construct
  source 1..44: mol_type = other DNA
atcagtatctcaggtaccgagctcttaccctaaccgagcctgcg

SEQ ID NO: 266 - DNA, 25 bp, synthetic construct
  source 1..25: mol_type = other DNA
tcttctaataaggggatcttgaagt

SEQ ID NO: 267 - DNA, 25 bp, synthetic construct
  source 1..25: mol_type = other DNA
cgctgttgcggatcatctccagcgt

SEQ ID NO: 268 - DNA, 47 bp, synthetic construct
  source 1..47: mol_type = other DNA
cgcgcggatcttccagagattgggcccgttgggctgccattttgcga

SEQ ID NO: 269 - DNA, 47 bp, synthetic construct
  source 1..47: mol_type = other DNA
aagcttgacgtccaggtgcctcttaagaactctgtttccttattttg

SEQ ID NO: 270 - DNA, 47 bp, synthetic construct
  source 1..47: mol_type = other DNA
cacgctagcggatccgagctcggtaccgaacatttatatgtataaat

SEQ ID NO: 271 - DNA, 47 bp, synthetic construct
  source 1..47: mol_type = other DNA
cgtgcggacggcaagctgctacagctgacagccagcgctggcagtaa

SEQ ID NO: 272 - DNA, 25 bp, synthetic construct
  source 1..25: mol_type = other DNA
tgaattagaactcggtacgcgcgga

SEQ ID NO: 273 - DNA, 25 bp, synthetic construct
  source 1..25: mol_type = other DNA
tagagaataggaacttcgaactgca

SEQ ID NO: 274 - DNA, 25 bp, synthetic construct
  source 1..25: mol_type = other DNA
aacttcgaagcagctccagcctaca

SEQ ID NO: 275 - DNA, 25 bp, synthetic construct
  source 1..25: mol_type = other DNA
atgattacgccaagtttgcacgcct

SEQ ID NO: 276 - DNA, 23 bp, synthetic construct
  source 1..23: mol_type = other DNA
tcgggtgtgacccaggcgcaagt

SEQ ID NO: 277 - DNA, 25 bp, synthetic construct
  source 1..25: mol_type = other DNA
cgccctgagtgcttgcggcagcgtg

SEQ ID NO: 278 - DNA, 42 bp, synthetic construct
  source 1..42: mol_type = other DNA
aggaaacagaccatggaattcatgtctgacgacatgtctatg

SEQ ID NO: 279 - DNA, 41 bp, synthetic construct
  source 1..41: mol_type = other DNA
tccgccaaaacagccaagcttttactcatcttcaagataag

SEQ ID NO: 280 - DNA, 25 bp, synthetic construct
  source 1..25: mol_type = other DNA
tcactgcataattcgtgtcgctcaa

SEQ ID NO: 281 - DNA, 25 bp, synthetic construct
  source 1..25: mol_type = other DNA
cgctactgccgccaggcaaattctg

SEQ ID NO: 282 - DNA, 41 bp, synthetic construct; identical to SEQ ID NO: 23
  source 1..41: mol_type = other DNA

SEQ ID NO: 283 - DNA, 44 bp, synthetic construct
  source 1..44: mol_type = other DNA
atcagtatctcaggtaccgagctcttactcatcttcaagataag

SEQ ID NO: 284 - DNA, 25 bp, synthetic construct
  source 1..25: mol_type = other DNA
tcttctaataaggggatcttgaagt

SEQ ID NO: 285 - DNA, 25 bp, synthetic construct
  source 1..25: mol_type = other DNA
cgctgttgcggatcatctccagcgt

SEQ ID NO: 286 - DNA, 48 bp, synthetic construct
  source 1..48: mol_type = other DNA
cgcgcggatcttccagagattgggcccgtgatctcttcgatgtctacc

SEQ ID NO: 287 - DNA, 47 bp, synthetic construct
  source 1..47: mol_type = other DNA
aagcttgacgtccaggtgcctcttaagagcgaacctcaaattatttt

SEQ ID NO: 288 - DNA, 47 bp, synthetic construct
  source 1..47: mol_type = other DNA
cacgctagcggatccgagctcggtaccgtcctgtgttacttgaatcc

SEQ ID NO: 289 - DNA, 47 bp, synthetic construct
  source 1..47: mol_type = other DNA
cgtgcggacggcaagctgctacagctggccagagcagtgatttccga

SEQ ID NO: 290 - DNA, 25 bp, synthetic construct
  source 1..25: mol_type = other DNA
tgaattagaactcggtacgcgcgga

SEQ ID NO: 291 - DNA, 25 bp, synthetic construct
  source 1..25: mol_type = other DNA
tagagaataggaacttcgaactgca

SEQ ID NO: 292 - DNA, 25 bp, synthetic construct
  source 1..25: mol_type = other DNA
aacttcgaagcagctccagcctaca

SEQ ID NO: 293 - DNA, 25 bp, synthetic construct
  source 1..25: mol_type = other DNA
atgattacgccaagtttgcacgcct

SEQ ID NO: 294 - DNA, 22 bp, synthetic construct
  source 1..22: mol_type = other DNA
gatgtcaggagactgtttgccg

SEQ ID NO: 295 - DNA, 25 bp, synthetic construct
  source 1..25: mol_type = other DNA
cgccctgagtgcttgcggcagcgtg

SEQ ID NO: 296 - DNA, 47 bp, synthetic construct; identical to SEQ ID NO: 109
  source 1..47: mol_type = other DNA

SEQ ID NO: 297 - DNA, 47 bp, synthetic construct; identical to SEQ ID NO: 110
  source 1..47: mol_type = other DNA

SEQ ID NO: 298 - DNA, 47 bp, synthetic construct; identical to SEQ ID NO: 111
  source 1..47: mol_type = other DNA

SEQ ID NO: 299 - DNA, 47 bp, synthetic construct; identical to SEQ ID NO: 112
  source 1..47: mol_type = other DNA

SEQ ID NO: 300 - DNA, 25 bp, synthetic construct
  source 1..25: mol_type = other DNA
tgaattagaactcggtacgcgcgga

SEQ ID NO: 301 - DNA, 25 bp, synthetic construct
  source 1..25: mol_type = other DNA
tagagaataggaacttcgaactgca

SEQ ID NO: 302 - DNA, 25 bp, synthetic construct
  source 1..25: mol_type = other DNA
aacttcgaagcagctccagcctaca

SEQ ID NO: 303 - DNA, 25 bp, synthetic construct
  source 1..25: mol_type = other DNA
atgattacgccaagtttgcacgcct

SEQ ID NO: 304 - DNA, 25 bp, synthetic construct
  source 1..25: mol_type = other DNA
cggtcgaaatcacggatgatgtagt

SEQ ID NO: 305 - DNA, 25 bp, synthetic construct
  source 1..25: mol_type = other DNA
cgccctgagtgcttgcggcagcgtg

SEQ ID NO: 306 - DNA, 41 bp, synthetic construct
  source 1..41: mol_type = other DNA
aggaaacagaccatggaattcatgtatatttattggatttt

SEQ ID NO: 307 - DNA, 41 bp, synthetic construct
  source 1..41: mol_type = other DNA
tccgccaaaacagccaagctttcaggcaagtttcaccatga

SEQ ID NO: 308 - DNA, 25 bp, synthetic construct
  source 1..25: mol_type = other DNA
tcactgcataattcgtgtcgctcaa

SEQ ID NO: 309 - DNA, 25 bp, synthetic construct
  source 1..25: mol_type = other DNA
cgctactgccgccaggcaaattctg

SEQ ID NO: 310 - DNA, 41 bp, synthetic construct; identical to SEQ ID NO: 23
  source 1..41: mol_type = other DNA

SEQ ID NO: 311 - DNA, 41 bp, synthetic construct
  source 1..41: mol_type = other DNA
agtatctcaggtaccgagctctcaggcaagtttcaccatga

SEQ ID NO: 312 - DNA, 25 bp, synthetic construct
  source 1..25: mol_type = other DNA
tcttctaataaggggatcttgaagt

SEQ ID NO: 313 - DNA, 25 bp, synthetic construct
  source 1..25: mol_type = other DNA
cgctgttgcggatcatctccagcgt

SEQ ID NO: 314 - DNA, 53 bp, synthetic construct
  source 1..53: mol_type = other DNA
cgcgcggatcttccagagattgggccccccggggataatgagaaacactgtca

SEQ ID NO: 315 - DNA, 47 bp, synthetic construct
  source 1..47: mol_type = other DNA
aagcttgacgtccaggtgcctcttaagtgtccttctcctgcaagaga

SEQ ID NO: 316 - DNA, 47 bp, synthetic construct
  source 1..47: mol_type = other DNA
cacgctagcggatccgagctcggtacctgaagacgctgcccgcgctg

SEQ ID NO: 317 - DNA, 53 bp, synthetic construct
  source 1..53: mol_type = other DNA
cgtgcggacggcaagctgctacagctggggcccttttatgcgcactgattacc

SEQ ID NO: 318 - DNA, 25 bp, synthetic construct
  source 1..25: mol_type = other DNA
tgaattagaactcggtacgcgcgga

SEQ ID NO: 319 - DNA, 25 bp, synthetic construct
  source 1..25: mol_type = other DNA
tagagaataggaacttcgaactgca

SEQ ID NO: 320 - DNA, 25 bp, synthetic construct
  source 1..25: mol_type = other DNA
aacttcgaagcagctccagcctaca

SEQ ID NO: 321 - DNA, 25 bp, synthetic construct
  source 1..25: mol_type = other DNA
atgattacgccaagtttgcacgcct

SEQ ID NO: 322 - DNA, 25 bp, synthetic construct
  source 1..25: mol_type = other DNA
gccagcaatccccagacaaaagcga

SEQ ID NO: 323 - DNA, 25 bp, synthetic construct
  source 1..25: mol_type = other DNA
cgccctgagtgcttgcggcagcgtg

SEQ ID NO: 324 - DNA, 47 bp, synthetic construct
  source 1..47: mol_type = other DNA
cgcgcggatcttccagagattgggcccccagagcagaaaatattggg

SEQ ID NO: 325 - DNA, 48 bp, synthetic construct
  source 1..48: mol_type = other DNA
aagcttgacgtccaggtgcctcttaagggttgttacctcgttaccttt

SEQ ID NO: 326 - DNA, 47 bp, synthetic construct
  source 1..47: mol_type = other DNA
cacgctagcggatccgagctcggtacccatggttaaagtttatgccc

SEQ ID NO: 327 - DNA, 45 bp, synthetic construct
  source 1..45: mol_type = other DNA
cgtgcggacggcaagctgctacagctgacccctgggttacggctt

SEQ ID NO: 328 - DNA, 25 bp, synthetic construct
  source 1..25: mol_type = other DNA
tgaattagaactcggtacgcgcgga

SEQ ID NO: 329 - DNA, 25 bp, synthetic construct
  source 1..25: mol_type = other DNA
tagagaataggaacttcgaactgca

SEQ ID NO: 330 - DNA, 25 bp, synthetic construct
  source 1..25: mol_type = other DNA
aacttcgaagcagctccagcctaca

SEQ ID NO: 331 - DNA, 25 bp, synthetic construct
  source 1..25: mol_type = other DNA
atgattacgccaagtttgcacgcct

SEQ ID NO: 332 - DNA, 20 bp, synthetic construct
  source 1..20: mol_type = other DNA
aagggctttttctgcgactt

SEQ ID NO: 333 - DNA, 25 bp, synthetic construct
  source 1..25: mol_type = other DNA
cgccctgagtgcttgcggcagcgtg

SEQ ID NO: 334 - DNA, 45 bp, synthetic construct
  source 1..45: mol_type = other DNA
cgcgcggatcttccagagattgggccccaacccaacgcgcgatgt

SEQ ID NO: 335 - DNA, 47 bp, synthetic construct
  source 1..47: mol_type = other DNA
aagcttgacgtccaggtgcctcttaagttttaccccttgtttgcagc

SEQ ID NO: 336 - DNA, 48 bp, synthetic construct
  source 1..48: mol_type = other DNA
cacgctagcggatccgagctcggtacctttcagaaatttaataatgcc

SEQ ID NO: 337 - DNA, 47 bp, synthetic construct
  source 1..47: mol_type = other DNA
cgtgcggacggcaagctgctacagctgttcgcctgtttaaagttaga

SEQ ID NO: 338 - DNA, 25 bp, synthetic construct
  source 1..25: mol_type = other DNA
tgaattagaactcggtacgcgcgga

SEQ ID NO: 339 - DNA, 25 bp, synthetic construct
  source 1..25: mol_type = other DNA
tagagaataggaacttcgaactgca

SEQ ID NO: 340 - DNA, 25 bp, synthetic construct
  source 1..25: mol_type = other DNA
aacttcgaagcagctccagcctaca

SEQ ID NO: 341 - DNA, 25 bp, synthetic construct
  source 1..25: mol_type = other DNA
atgattacgccaagtttgcacgcct

SEQ ID NO: 342 - DNA, 20 bp, synthetic construct
  source 1..20: mol_type = other DNA
tgcgttgtcccaccgatcca

SEQ ID NO: 343 - DNA, 25 bp, synthetic construct
  source 1..25: mol_type = other DNA
cgccctgagtgcttgcggcagcgtg

SEQ ID NO: 344 - DNA, 47 bp, synthetic construct
  source 1..47: mol_type = other DNA
cgcgcggatcttccagagattgggcccgccgccgcctgtagcgataa

SEQ ID NO: 345 - DNA, 47 bp, synthetic construct
  source 1..47: mol_type = other DNA
aagcttgacgtccaggtgcctcttaagtgcctcactcctgccgtgaa

SEQ ID NO: 346 - DNA, 47 bp, synthetic construct
  source 1..47: mol_type = other DNA
cacgctagcggatccgagctcggtacctgactttcgccggacgtcag

SEQ ID NO: 347 - DNA, 47 bp, synthetic construct
  source 1..47: mol_type = other DNA
cgtgcggacggcaagctgctacagctgcgtgcggttggcctggctgg

SEQ ID NO: 348 - DNA, 25 bp, synthetic construct
  source 1..25: mol_type = other DNA
tgaattagaactcggtacgcgcgga

SEQ ID NO: 349 - DNA, 25 bp, synthetic construct
  source 1..25: mol_type = other DNA
tagagaataggaacttcgaactgca

SEQ ID NO: 350 - DNA, 25 bp, synthetic construct
  source 1..25: mol_type = other DNA
aacttcgaagcagctccagcctaca

SEQ ID NO: 351 - DNA, 25 bp, synthetic construct
  source 1..25: mol_type = other DNA
atgattacgccaagtttgcacgcct

SEQ ID NO: 352 - DNA, 25 bp, synthetic construct
  source 1..25: mol_type = other DNA
cagcttgcagtcggttaaccaggac

SEQ ID NO: 353 - DNA, 25 bp, synthetic construct
  source 1..25: mol_type = other DNA
cgccctgagtgcttgcggcagcgtg

SEQ ID NO: 354 - DNA, 47 bp, synthetic construct; identical to SEQ ID NO: 166
  source 1..47: mol_type = other DNA

SEQ ID NO: 355 - DNA, 47 bp, synthetic construct
  source 1..47: mol_type = other DNA
aagcttgacgtccaggtgcctcttaaggatgaacctcggctaacaaa

SEQ ID NO: 356 - DNA, 48 bp, synthetic construct
  source 1..48: mol_type = other DNA
cacgctagcggatccgagctcggtaccctttatggattaacggtgact

SEQ ID NO: 357 - DNA, 47 bp, synthetic construct
  source 1..47: mol_type = other DNA
cgtgcggacggcaagctgctacagctttgctgccatgcagtaaccac

SEQ ID NO: 358 - DNA, 25 bp, synthetic construct
  source 1..25: mol_type = other DNA
tgaattagaactcggtacgcgcgga

SEQ ID NO: 359 - DNA, 25 bp, synthetic construct
  source 1..25: mol_type = other DNA
tagagaataggaacttcgaactgca

SEQ ID NO: 360 - DNA, 25 bp, synthetic construct
  source 1..25: mol_type = other DNA
aacttcgaagcagctccagcctaca

SEQ ID NO: 361 - DNA, 25 bp, synthetic construct
  source 1..25: mol_type = other DNA
atgattacgccaagtttgcacgcct

SEQ ID NO: 362 - DNA, 20 bp, synthetic construct
  source 1..20: mol_type = other DNA
gggatcagcaggggaaagag

SEQ ID NO: 363 - DNA, 25 bp, synthetic construct
  source 1..25: mol_type = other DNA
cgccctgagtgcttgcggcagcgtg